(19) United States
(12) Patent Application Publication
(10) Pub. No.: US 2026/0128176 A1
(43) Pub. Date: May 7, 2026

(54) SYSTEMS AND METHODS FOR IMPROVING LOCATION-BASED SOCIAL DETERMINANTS OF HEALTH

(71) Applicant: Konnected Technologies Inc., Englewood, CO (US)

(72) Inventors: Brittany Mary Katalenas, Englewood, CO (US); Bianca Jasmin, Colorado Springs, CO (US); Kooper James Kuehl, Greensburg, IN (US)

(21) Appl. No.: 19/376,034

(22) Filed: Oct. 31, 2025

Related U.S. Application Data
(60) Provisional application No. 63/715,176, filed on Nov. 1, 2024.

(51) Int. Cl. G16H 50/30; G16H 10/20

(52) U.S. Cl. CPC G16H 50/30; G16H 10/20

(57) ABSTRACT

This invention pertains to a computer system for evaluating and improving community health scores for a location. The system includes one or more resident devices configured to generate and display a resident evaluation with questions corresponding to social health factors. Resident evaluation data collected from the devices reflects at least one social determinant of health for the location. A remote system server communicates with the resident devices and external data sources via an external network. The server includes processors programmed to receive location data, receive resident evaluation data, and generate a community social health score based at least in part on both. The processors are further configured to determine at least one location improvement or community service to increase the score and to cause information about the improvement or service to be displayed on a location management platform through a graphical user interface.

### CROSS-REFERENCE TO RELATED APPLICATION

This application claims the benefit of priority to U.S. Provisional Patent Application No. 63/715,176, filed Nov. 1, 2024, entitled “SYSTEMS AND METHODS FOR ADVANCED LOCATION SOCIAL HEALTH MONITORING,” the entire content and disclosure of which is hereby incorporated herein by reference in its entirety.

### FIELD OF THE DISCLOSURE

The field of the invention relates generally to generating a social health score for a location and improving the social health score, and more specifically, systems and methods for measuring and monitoring social health data for a location within a community and improving the community social health score.

### BACKGROUND

Numerous challenges may exist for location managers, such as maintenance, resident well-being, and operating efficiency. Conventionally, location management is focused on streamlining specific tasks and measuring property performance utilizing financial models. However, while conventional systems can manage tangible elements of property management the conventional systems lack accountability, transparency, and the ability to evaluate the impact of social determinants of health on the location, the residents, and the community corresponding to the location. These limitations prevent the detection and enhancement of resident well-being, community engagement, and improved operational efficiencies through informed data-driven solutions.

Additionally, conventional approaches to property management and social health assessment are typically siloed by property type, with separate systems and methodologies for residential housing, healthcare facilities, educational institutions, government facilities, and other site classifications. This fragmentation prevents cross-sector comparison, limits data standardization, and impedes the development of comprehensive community health strategies that span multiple property types.

What is needed is a universal system capable of measuring both personal stability and community stability across any site or property type using standardized social determinants of health metrics, thereby enabling comparable assessment and intervention strategies regardless of whether the location is residential housing, a healthcare facility, an educational institution, a government facility, or any other site where individuals interact with their environment. In particular, there is a need for a system that can quantify the social health of a location within a community by generating social health scores that identify areas for improvement and recommend community services to enhance overall well-being. Conventional techniques often lack this capability and may introduce inefficiencies, encumbrances, ineffectiveness, or other drawbacks, thereby limiting their ability to provide actionable insights into both individual and community health outcomes.

### BRIEF DESCRIPTION

The present embodiments may relate to systems and methods for evaluating and improving social health factors for a location. The social health factors are associated with the location, the residents of the location, and the community corresponding to the location. The system may include a remote system server, a location management platform, an improvement recommender server, and one or more resident devices associated with the location. The location management platform may be installed within the location and may be configured to receive location data from the one or more resident devices via a location network. The location data may reflect an aspect of social health of one or more residents of the location. The system may serve as a universal framework appliable across any location type, including but not limited to residential properties, healthcare facilities, educational institutions, government facilities, correctional facilities, homeless shelters, senior living facilities, and community centers, thereby enabling standardized measurement of both personal stability and community stability regardless of specific property classification.

In one aspect, a computer system for evaluating and improving community health scores for a location is provided. The computer system includes: one or more resident devices corresponding to the location; a resident evaluation generated on each of the resident devices, the resident evaluation including one or more evaluation questions to collect resident evaluation data from the one or more resident devices, wherein each of the one or more evaluation questions correspond to one or more social health factors, the resident evaluation data reflecting at least one social determinant of health corresponding to each of the resident devices corresponding to the location; and a remote system server configured to communicate with the one or more resident devices and one or more external data sources corresponding to the location via an external network. The remote system server includes one or more processors programmed to: (i) receive a first element of location data from the external data source relating to the location; (ii) receive the resident evaluation data; (iii) generate a community social health score for the location based at least in part on the location data and the resident evaluation data; (iv) determine at least one location improvement or community service to improve the community health score; and (v) cause to be displayed, to a location management platform for the location via a graphical user interface, information about the at least one location improvement and community service.

In another aspect, a location management computer platform for collecting resident data and providing the data to a location management service provider is provided. The platform includes a resident evaluation, at least one external data source, and one or more processors in communication with memory. The processors are programmed to: (i) generate a plurality of evaluation questions for the resident evaluation, each evaluation question corresponding to at least one social health factor; (ii) receive resident evaluation data from a select resident device; (iii) receive location data from at least one location device or the external data source; (iv) generate a community social health score for the location based on the resident evaluation data and the location data; and (v) generate and post an electronic notification to a dashboard accessible by the location management service provider, the notification detailing an improvement identified to increase the community social health score of the location.

In another aspect, a computer-implemented method for evaluating and improving community health scores for a location is provided. The method is carried out using a computer system having one or more processors and includes: (i) generating, for display on one or more resident computing devices associated with the location, a resident evaluation including one or more evaluation questions, each evaluation question corresponding to at least one social health factor; (ii) receiving resident evaluation data from the one or more resident computing devices, the resident evaluation data reflecting at least one social health factor associated with the location; (iii) receiving location data from at least one external data source; and (iv) generating a community social health score for the location based at least in part on the resident evaluation data and the location data.

The present embodiments may further relate to systems and methods that provide a universal framework for measuring and improving social health across any type of location, site, or property where individuals reside, work, receive services, or congregate. While exemplary embodiments may be described with reference to residential properties such as apartment buildings or multi-family housing, the skilled artisan will understand that the disclosed systems and methods are equally applicable to a wide range of location types, including but not limited to: Healthcare and Medical Facilities: hospitals, medical centers, rehabilitation facilities, mental health treatment facilities, substance abuse treatment centers, dialysis centers, and outpatient clinics, wherein the systems may measure patient stability, access to care, treatment outcomes, and community health impact; Educational Institutions: schools, universities, colleges, vocational training centers, and educational programs, wherein the systems may measure student well-being, educational access, social support systems, and community engagement; Government and Public Facilities: government office buildings, public service facilities, municipal facilities, and government program sites (such as Medicaid service locations, social service offices, or public assistance facilities), wherein the systems may measure citizen access to services, program effectiveness, and public health outcomes; Transitional and Support Housing: homeless shelters, transitional housing facilities, domestic violence shelters, emergency housing, and supportive housing programs, wherein the systems may measure resident stability, service access, safety, and transition success rates; Senior and Specialized Care: senior living facilities, assisted living facilities, nursing homes, memory care facilities, and specialized care facilities, wherein the systems may measure resident health, care quality, social engagement, and overall well-being; Correctional and Justice Facilities: correctional facilities, detention centers, halfway houses, and reentry programs, wherein the systems may measure inmate or participant well-being, program effectiveness, and reintegration success; Community and Congregate Facilities: community centers, religious facilities, recreational facilities, and any other locations where individuals congregate for social, educational, or community purposes, wherein the systems may measure community engagement, social cohesion, and collective well-being. In each of these contexts, the systems and methods described herein may measure both personal stability (the well-being, safety, and social health of individual persons associated with the location) and community stability (the aggregate social health, safety, and well-being of the community surrounding or associated with the location).

The social determinants of health framework, including housing quality and stability, economic stability, access to health services, neighborhood and built environment, and social and community context, may apply universally across all location types, though the specific metrics, weightings, and interventions may be customized based on location classification. For example, in a healthcare facility context, the “location social health score” may emphasize access to medical care, treatment outcomes, and patient safety, while the “community social health score” may reflect the facility's impact on surrounding community health outcomes. In an educational facility context, the “location social health score” may emphasize educational access, student safety, and support resources, while the “community social health score” may reflect educational attainment levels and community engagement. In a government facility or program context (such as a Medicaid program), the “location” may represent a service delivery site or program jurisdiction (such as a state or county), the “location social health score” may reflect program accessibility and service quality, and the “community social health score” may reflect health outcomes and social stability across the served population. The universal applicability of the disclosed systems and methods may provide for cross-sector comparison, standardized data collection across diverse location types, and comprehensive community-wide health assessment by aggregating data from multiple location types within a geographic area. This universality represents a significant advance over conventional siloed approaches that treat each facility or property type in isolation.

The present embodiments may further relate to a universal computer system for evaluating and improving community health scores for any type of location, site, or property, including but not limited to residential properties, healthcare facilities, educational institutions, government facilities, transitional housing, senior living facilities, and community centers. The system can provide a standardized framework for measuring both personal stability and community stability across any location type using social determinants of health metrics. The system may include one or more resident devices (or user devices associated with individuals at any such location) configured to generate and display an evaluation with questions corresponding to social health factors. Evaluation data collected from the devices may reflect at least one social determinant of health for the location. A remote system server can communicate with the resident devices and external data sources via an external network. The server may include processors programmed to receive location data, receive resident evaluation data, and generate a community social health score based at least in part on both. The processors may be further configured to determine at least one location improvement or community service to increase the score and to cause information about the improvement or service to be displayed on a location management platform through a graphical user interface. The system's universal applicability may provide for the standardized assessment and intervention across diverse location types and facilitates cross-sector comparison and community-wide health planning.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple figures are designated with consistent reference numerals.

There are shown, in the drawings, arrangements which are presently discussed herein. However, it should be understood that the present embodiments are not limited to the precise arrangements and/or instrumentalities shown herein.

FIG. 1 illustrates an exemplary system for monitoring and analyzing social heath factors of a location, in accordance with at least one embodiment of this disclosure.

FIG. 2 illustrates an expanded location monitoring and evaluation (“LME”) system that may be used for evaluating a location and the factors associated with the location and for providing solutions to improve those factors, in accordance with the present disclosure.

FIG. 3 illustrates exemplary resident devices that may be used with the system shown in FIG. 1 and the system shown in FIG. 2.

FIG. 4 illustrates an exemplary computer system for implementing the systems shown in FIGS. 1 and 2 performing the process shown in FIG. 7.

FIG. 5 depicts an exemplary configuration of a user computer device, in accordance with one embodiment of the present disclosure.

FIG. 6 depicts an exemplary configuration of a server computer device, in accordance with one embodiment of the present disclosure.

FIG. 7 depicts a flow chart of an exemplary computer-implemented process for evaluating and improving aspects of community health for a location in FIG. 1 using the systems shown in FIGS. 1 and 2.

FIGS. 8A-8E illustrate graphs of different scores in different categories based upon the systems and methods described herein.

FIG. 9 illustrates a map of different residents and/or facilities and their corresponding categories, in accordance with at least one embodiment.

The figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

The present embodiments may relate to, inter alia, systems and methods for monitoring a social health of a location and/or resident to determine and provide solutions for improving social health factors of the location. The social health factors may correspond to the social determinants of health of the resident and/or location. The social health factors may be represented by the system through the generation of a social health score. The social health score can correspond to the social health of the location, the resident, and/or the community. For example, the social health score can correspond to the social health of a resident associated with a location. Additionally, the social health score can correspond to the social health of a community associated with the location. Accordingly, the platform enables the evaluation of the overall social health impact of the location on the surrounding area. As used herein, resident, tenant, citizen, or community member may mean an individual associated with the location, house, condominium, apartment, or any other community structure. In one exemplary embodiment, a location monitoring and evaluation (LME) system coordinates a location management ecosystem. The location management ecosystem includes the LME system and a location management platform for collecting location data and identifying location improvement and community services. The location management ecosystem is configured to collaborate between residents and location managers to coordinate community services, location maintenance, location improvements, and social health improvements. The location social health monitoring and analysis system acts as the orchestrator of this ecosystem. The location social health monitoring and analysis system may be configured to derive unique insights via artificial intelligence/machine learning (AI/ML). The location social health monitoring and analysis system is configured to provide social health understanding, analysis, and improvement capabilities to the locations, residents, and communities within the ecosystem.

The field of the invention relates generally to generating a social health score for a location and improving the social health score, and more specifically, systems and methods for measuring and monitoring social health data for a location within a community and improving the community social health score. As used herein, a “location” may include any type of site or property including, but not limited to, residential properties (such as apartment buildings, multi-family housing, condominiums, single-family homes, or affordable housing), institutional facilities (such as hospitals, healthcare facilities, rehabilitation centers, or mental health facilities), educational facilities (such as schools, universities, or training centers), government facilities, correctional facilities, homeless shelters, transitional housing, senior living facilities, assisted living facilities, community centers, or any other site where individuals reside, work, receive services, or congregate. The systems and methods described herein can provide a universal framework for measuring both personal stability and community stability across any such location type, enabling stakeholders to assess and improve social determinants of health regardless of the specific property or site classification.

As used herein, Social Determinants of Health (SDOH) refer to the non-medical factors that influence health outcomes, including the conditions in which people live, work, and interact. For the methodology described herein, SDOH encompass key elements that directly impact the well-being of residents within a property. These key elements may include, but are not limited to, housing quality and stability, economic stability, access to health services, neighborhood and build environment, and social and community context.

Housing Quality and Stability refers to the safety, affordability, and condition of the living environment. Economic Stability refers to access to employment, income levels, and financial security, including the ability to afford housing and basic necessities. Access to Health Services includes the availability and accessibility of healthcare services within the community. Neighborhood and Built Environment covers the safety, infrastructure, and access to transportation, public spaces, and community resources. Social and Community Context refers to relationships, social networks, and support systems available within the property and community that influence mental and emotional well-being. In the LME system, these factors are systematically measured and tracked in relation to property conditions, influencing both individual health outcomes and overall community health.

In some embodiments, the social health score may also be known as Resident Success. This score breaks down properties and operations into four different categories BK Seal Metrics: Resident Success, Developing or Progressing or Stable or Solid (Progressive), Distressed (Developing), and Hazard (Passable). If the score exceeds a first threshold, such as greater than 86, this is considered the Success category and indicates exemplary operations and outstanding health outcomes, setting a high standard for resident well-being. If the score is between the first and second thresholds, such as between 85 and 66, this is considered the Developing or Progressing or Stable or Solid (Progressive) category. This indicates reliable operations with consistently strong health outcomes, showing effective management but not at the highest excellence level. If the score is between the second and third thresholds, such as between 65 and 36, this indicates the Distressed (Developing) category. This indicates moderate operations with improving health outcomes. If the score is below the third threshold, such as below 36, this indicates the Hazard (Passable) category. This indicates significant habitability concerns affecting the health of residents and the broader community, requiring immediate attention.

The LME system collects location data about the location, which may include resident social health data. Resident social health data may include various types of data that may represent social health factors or may reflect the social health impact of a condition on the social health score. For example, the resident social health data may include an evaluation provided to the resident device, where the evaluation is generated to collect social health data from the resident such that each evaluation question corresponds to one or more social health factors. In various embodiments, the system curates a specialized evaluation to collect social health data from residents of a location. In some embodiments, the evaluation provides a scale to respond to each of the questions. The resident evaluation data may then be used to analyze and extract social health factors associated with the resident and/or the location. In various embodiments, the system may generate weights for each of the evaluation questions based on the plurality of social health factors. For example, a question may be associated with a first social health factor at a first weight and a second social health factor at a second weight. By weighting the questions, the system can extract the social health data with the minimum amount of computer resources. Accordingly, the system generates the minimum number of questions required to the social health data from the resident, minimizing the computer resources required to generate the social health score and provide the solutions to improve the social health score. Additionally, by weighting the questions, the system minimizes burden on the resident to provide the social health data. In some embodiments, the evaluation can be a survey.

In various embodiments, the system provides the evaluation to a resident device. The evaluation can also be administered physically and entered into the LME system. In various embodiments, the resident interacts with the resident device to provide the resident data. In various embodiments, the system generates the resident evaluation data questions. The resident evaluation data questions can be generated by the machine learning module. In various embodiments, the evaluation includes a plurality of resident evaluation questions based, at least in part, on the location data. The question can be correlated to multiple social health factors. The social health factors can correspond to the social determinants of health (SDOH) outlined by government agencies. For example, the evaluation can include 28 indicators per domain across 5 SDOH domains. The resident evaluation can be generated to collect information from the residents based on a resident profile. For example, the evaluation may answer questions that indicate the social health of a resident of a location. The system can process the evaluation data to identify social health factors associated with the resident and social health factors associated with the location. Accordingly, the resident evaluation data can be processed by the system to generate the location social health score and the resident social health score, and the community social health score.

In various embodiments, the system supports multi-language functionality for resident evaluations, enabling each evaluation question to be presented in a plurality of languages. For example, the system may store a translation table for each evaluation question such that a resident device automatically displays the question in the preferred language of the resident, including but not limited to English, Spanish, or French. In some embodiments, the translation may be performed on a per-question basis, allowing the system to dynamically substitute the appropriate language version of each question while maintaining consistent mapping to the underlying social health factors.

In various embodiments, the system provides configurable evaluation capabilities that allow location managers to tailor the resident evaluation to the needs of a particular property or community. For example, the evaluation may include Likert scale questions ranging from “strongly disagree” to “strongly agree,” enabling standardized measurement of resident perceptions across multiple social health factors. In other embodiments, the evaluation may include additional question types such as yes/no questions or free-text responses, thereby capturing both quantitative and qualitative resident feedback. Each evaluation question may be mapped to one or more Social Determinants of Health (SDOH) categories, ensuring that the collected data is systematically aligned with recognized health and community well-being metrics.

In various embodiments, each evaluation session is assigned a unique anonymous identifier generated by the system. This identifier allows the system to track and aggregate responses without linking them to personally identifiable information, thereby preserving resident privacy while still enabling longitudinal analysis of evaluation data. In some embodiments, the system may further generate a quick response (QR) code corresponding to a particular evaluation instance, allowing residents to access the evaluation directly by scanning the QR code with a mobile device. This approach simplifies distribution and increases participation rates while maintaining anonymity.

In various embodiments, evaluation respondents may not be required to log in to complete an evaluation. Instead, the unique anonymous identifier and QR code access mechanism enable responses to be collected securely without requiring resident credentials. To further improve usability, the evaluation interface may employ emoji-based visual indicators for response options, providing an intuitive and accessible way for residents to express their feedback across different languages and literacy levels. The system may allow location managers to enable or disable evaluations on a per-property basis, ensuring that only relevant evaluations are active for a given location at any time.

In some embodiments, the system may be further configured with data integrity features that allow evaluation data to be replayed and filtered to remove malicious or inaccurate responses. For example, the LME server may store a log of raw evaluation submissions and enable reprocessing of those submissions to identify anomalies such as duplicate entries, inconsistent answer patterns, or responses outside of expected ranges. The system may then filter out such responses before computing the location social health score, the resident social health score, or the community social health score. This replay and filtering capability ensures that the resulting scores and recommendations are based on reliable data, thereby improving the accuracy of the system's analysis and the effectiveness of the proposed location improvements and community services.

In various embodiments, the system may further include an alerting module configured to generate notifications when social health scores or related factors cross configurable thresholds. For example, a location manager may define threshold values for one or more social health factors or for the overall community social health score. When the monitored values fall below or exceed the defined thresholds, the system may automatically generate and transmit an alert to designated recipients. The alerts may be delivered through multiple communication channels, including email and short message service (SMS) notifications, and may be customized on a per-user or per-property basis. In some embodiments, the system may also allow administrators to establish default alert configurations for all users, while permitting individual users to adjust their own preferences.

In various embodiments, the social health scores can be monitored over time to predict a disruption to the social health of the resident. In some embodiments, the system monitors the social health score over time to preemptively identify indicators associated with a decrease in the social health score. By continuously monitoring the social health of the location, the system can predict a condition affecting the social health score and provide solutions to mitigate the impact to the social health score. For example, the system may detect a condition that would affect the social score such as changes in social dynamics (e.g., community engagement, access to social services, inclusion, or the like) or from man-made challenges or other inherent social risks to a community (e.g., isolation due to lack of communal spaces, crime or lack of safety measures, inadequate social support systems or healthcare, education, or the like). The system also identifies other conditions that may positively or negatively impact the social health score. The LME system may also collect data about different attributes of the location, such as, but not limited to, power usage, water usage, current temperatures, maintenance history, as well as current and future social health conditions.

The LME system may collect some location data from external sources (e.g., publicly available data, such as historical community information or community statistics for the area, or the like) or from location sources (e.g., data gathered from sensors, a location database, or networked devices within and/or around the location). The location manager may also provide the LME system with access to data from different monitors and other devices associated with the location, such as Internet of Things (IoT) devices. The IoT devices may provide their data directly and/or provide data through servers associated with and in communication with the IoT devices.

The LME system may then analyze the provided data to, for example, generate the location social health score. The location social health score represents the social health impact of the location. Accordingly, the location social health score represents the underlying impact of the condition of the location on social health factors. Additionally, the LME system can generate the resident social health score. The resident social health score includes the resident data and the location social health score. Accordingly, the resident social health score represents the social health of the resident with regards to the impact of the location. In various embodiments, the LME system generates a community social health score. The community social health score can reflect a plurality of resident social health scores and location social health scores. Accordingly, the location social health score, the resident social health score, and the community social health score demonstrate the overall social health impact of the location.

In various embodiments, the LME server can process the provided data to generate solutions to improve social health factors of a location to improve the social score. The provided solutions may include identifying community services to improve resident social health, and ongoing historical data collection for ongoing community social health monitoring. As such, locations may include a location management platform that is configured (e.g., on a communication network) to communicate with various sensors, residents, and other community stakeholders to relay community social health data to a remote system server for various uses discussed herein.

In one exemplary embodiment, the LME system may include a community social health model engine that may capture resident social health data from resident devices and provide improvements to the location manager, or perhaps community services (e.g., social services, emergency services), when current or detected community social health improvements to the location are detected. For example, the resident device may be configured to provide a user interface for a resident to perform an evaluation to collect resident social health data to identify solutions to improve the social health factors for the location such as enhancements in community infrastructure (e.g., development of public spaces, community centers, public transportation, or the like) and community programs (e.g., programs for social health, safety initiatives to reduce crime, investment in local healthcare and education, community services or utilities, or the like).

The community social health model engine may regularly evaluate such data and may provide, for example, evaluations of potential improvements to the location (e.g., articulating the source and nature of the improvement to the location), recommendations to the location manager for community or resident social health improvement (e.g., detailing how the community or resident social health may be improved), or alerting for conditions detected within the location (e.g., transmitting an alert to a device or location management platform of the location manager upon detection of a condition impacting the social health factors. In some embodiments, the LME system may provide a community health score to the location manager, which may be used to identify areas of improvement or risk reduction that may impact the community social health of the location. The community social health score includes location data, resident data, and community data. As such, the LME system helps identify and improve the social health impact on the surrounding area to improve the social health for the location, the residents, and the community. The LME system may analyze the location data to determine one or more issues with the location to notify the location manager or resident about. In one exemplary embodiment, the LME system may include a location evaluation engine that may evaluate location data from external or location sources to evaluate various social health factors associated with the location. The LME system may use numerous data points to evaluate social health factors impacting a location and may compute a location social health score or various health scores for the location.

Such social health scores may be used, for example by a location manager, to evaluate social health of the location and its residents, to identify improvements to the location to increase community social health, or to connect residents with services to improve their overall health. For example, the location evaluation engine may generate a community social health score for the location based upon factors such as, inter alia, the community engagement levels, the frequency and diversity of social events, the availability and accessibility of public services within the area, the incidence and response to social disparities encountered by residents, or the presence and effectiveness of community support structures.

The LME system may also be in communication with one or more community service providers that provide access to and matching with organizations that provide products and/or services that may improve the community social heath score and improve the impact of the location social health score on the community social health score. The community service providers may also provide access to and matching with companies to remediate and/or improve conditions impacting the community social health that are currently occurring at the location. One of the goals is to help the location manager to make their location better, healthier, and more sustainable. Other examples of products and/or services provided by the community service providers include, but are not limited to mental health support services, educational programs, community safety initiatives, social integration activities, and/or access to healthcare and wellness programs.

The location evaluation engine may also generate a social health score for the location based upon factors such as, inter alia, the degree of community engagement and social support networks, frequency of community-held events and activities, effectiveness of local governance and public service provision, or the presence and utilization of public spaces and community centers. The location evaluation engine may generate a community health score from the location based upon factors such as, inter alia, various resident behaviors or demographics, a number of actionable notifications generated by the system, and social health condition resolution responsiveness. Such scores may be used to evaluate social health factors, indicating more or less impact on the social health of the location and the residents associated within the location and, as such, may impact community social health factors for the location.

In the exemplary embodiment, the location management server provides the residents access to the community service providers, while using ML (machine learning) and AI (artificial intelligence) to determine improvement and community services most relevant to the resident based on the analysis of social health. In at least some embodiments, the location management server determines different attributes and/or conditions of the location based on the location data provided from the resident devices and/or the external data sources. The location evaluation server may use the different attributes and/or conditions of the location to build a digital location profile of the location. Then the location management server may use the digital location profile and the current location data to determine a community social health score. The community social health score is an abstraction that provides a value for the current condition of the location and based on the aggregate of different attributes and/or conditions of the residents and the location. In the exemplary embodiment, the home community health score is a mix of characteristics to provide a holistic view regarding the well-being of a community associated with a location. Elements about the location, as well as the surrounding environment, are taken into consideration as part of the calculation for the community health score. The location management server may also determine/calculate a resident profile score that is an aggregate score displayed to the user, such as through a dashboard, which calculates subcategories of individual and community social health risks for a resident based on resident data and external data. The resident profile score may be provided through a mobile app that allows a user to search for a location and see a dynamic “Location Profile Dashboard” that includes a personalized community health score, recommendations, and service providers.

While various examples provided herein describe application of the LME system to various aspects of homes, the systems and methods described herein may also be used for performing other analysis, such as public spaces, businesses, non-profits, shelters, municipal locations, and/or other locations and/or items.

In another embodiment, the system as described herein may include additional features such as multi-domain question mapping. In this configuration, a single evaluation question may be mapped to multiple Social Determinants of Health (SDOH) categories, with dynamic weighting applied across domains to reduce evaluation length while maintaining accuracy and predictive power.

In another embodiment, the system as described herein may include additional features such as a React-based frontend architecture for delivering responsive, component-driven user interfaces. The frontend may integrate standard React ecosystem libraries to support multilingual rendering, evaluation forms, and dashboards.

In another embodiment, the system as described herein may include additional features such as a Spring Boot backend implemented in Java/Kotlin. The backend may provide stateless APIs, enforce authentication and authorization, and manage evaluation, analytics, and user management logic.

In another embodiment, the system as described herein may include additional features such as MongoDB for data persistence. The database may support landlord-property-evaluation-response structures, enforce tenant-level isolation, and optimize queries filtered by user permissions.

In another embodiment, the system as described herein may include additional features such as AWS-based hosting infrastructure. ECS Fargate may provide compute, CloudFront may serve as a CDN, S3 may host static content, and AWS Secrets Manager may secure credentials and API keys.

In another embodiment, the system as described herein may include additional features such as end-to-end encryption between client and server. JWT cookie-based authentication may be used for all API requests, with tokens stored outside of browser localStorage/sessionStorage.

In another embodiment, the system as described herein may include additional features such as granular permission scoping for different user types. Queries may be filtered by role so that users only access data within their assigned properties or organizations.

In another embodiment, the system as described herein may include additional features such as complete anonymization of evaluation responses. Responses may be linked only to properties, not individuals, with unique anonymous session IDs generated for each evaluation.

In another embodiment, the system as described herein may include additional features such as three distinct user types with different access levels. Admins may configure customers and evaluations, landlords may manage properties and dashboards, and tenants may respond anonymously via unique URLs or QR codes.

In another embodiment, the system as described herein may include additional features such as multi-language evaluation support. Evaluations may currently be delivered in English and Spanish, with expansion to French and other languages supported through per-question translation.

In another embodiment, the system as described herein may include additional features such as configurable Likert-scale questions. The system may also support yes/no and free-text responses, with questions mapped to Social Determinants of Health (SDOH) categories.

In another embodiment, the system as described herein may include additional features such as QR code generation for evaluation distribution. Each property/evaluation combination may generate a unique URL path containing randomized anonymous identifiers, secured under a unified HTTPS domain.

In another embodiment, the system as described herein may include additional features such as emoji-based visual indicators for evaluation responses. These indicators may improve accessibility and usability across languages and literacy levels.

In another embodiment, the system as described herein may include additional features such as the ability to enable or disable evaluations per property. Properties may be explicitly linked to evaluations through evaluation assignments in the hierarchical data model.

In another embodiment, the system as described herein may include additional features such as aggregate scoring and breakdowns by SDOH categories. Total response counts and overall score calculations may be displayed alongside dashboards for context.

In another embodiment, the system as described herein may include additional features such as dashboard visualization of evaluation responses. Graphical interfaces may include trend lines, heat maps, and categorical breakdowns for real-time interpretation.

In another embodiment, the system as described herein may include additional features such as replay and filtering of evaluation data to remove malicious responses. The system may detect anomalies such as duplicate entries or suspicious patterns while preserving anonymity.

In another embodiment, the system as described herein may include additional features such as change-over-time tracking. Historical baselines may be compared to current scores to measure program impact and identify trends.

In another embodiment, the system as described herein may include additional features such as configurable alert thresholds. Alerts may be delivered via email or SMS when metrics cross defined values, with per-user and per-customer preferences.

In another embodiment, the system as described herein may include additional features such as support for granular data analysis per property. Property-specific dashboards may allow filtering by attributes such as building type, size, or demographics.

In another embodiment, the system as described herein may include additional features such as file handling capabilities. The system may support file uploads, CSV and Excel processing via Papaparse and SheetJS, and import/export of structured data.

In another embodiment, the system as described herein may include additional features such as a hierarchical data model. Landlords may manage multiple properties, each property may enable multiple evaluations, and responses may generate unique anonymous session IDs.

In another embodiment, the system as described herein may include additional features such as custom property attributes and metadata. Multiple users per customer organization may be supported with role-based access to properties and evaluations.

In another embodiment, the system as described herein may include additional features such as URL routing with language-specific paths. Different URL paths may be generated for different languages to direct respondents to localized evaluation versions.

In another embodiment, the system as described herein may include additional features such as elastic cloud scaling. The platform may handle multiple concurrent users per customer organization and optimize database queries for performance.

In another embodiment, the system as described herein may include additional features such as integration with property management platforms. API endpoints may be exposed for external system access, with webhook/event-driven notifications supported.

In another embodiment, the system as described herein may include additional features such as API authentication for government entities. Entities such as Boulder County may securely access anonymized aggregate data through authenticated APIs.

In another embodiment, the system as described herein may include additional features such as configuration flexibility. Customers may upload logos for branding, customize dashboards, and configure alerts through drop-down selections.

In another embodiment, the system as described herein may include additional features such as authentication flows with temporary passwords. New customers may receive emailed credentials, be forced to reset on first login, and use self-service password reset.

In another embodiment, the system as described herein may include additional features such as audit trails for compliance. Each action may be tied to a specific user, with password-protected admin access and auditable user actions.

In another embodiment, the system as described herein may include additional features such as multi-tenancy support. Landlords, non-profits, and government entities may be modeled, with properties mapped to organizational hierarchies such as Medicaid states.

In another embodiment, the system as described herein may include additional features such as real-time predictive analytics. Machine learning models may forecast changes in social health conditions and display results through dashboards.

In another embodiment, the system as described herein may include additional features such as evaluation bombing detection. The system may flag abnormal submission rates (e.g., 1000+ in 10 minutes) and retroactively filter fraudulent data.

In another embodiment, the system as described herein may include additional features such as anonymous fraud detection. Pattern recognition may identify suspicious responses while preserving complete anonymity.

In another embodiment, the system as described herein may include additional features such as government integration architecture. County-wide implementations may be supported with milestone-based activation and aggregate reporting at multiple levels.

In another embodiment, the system as described herein may include additional features such as advanced scoring methods. Cross-domain correlation discovery, market normalization, and composite indices may refine accuracy and comparability.

In another embodiment, the system as described herein may include additional features such as certification seal generation. Automated seals may be produced in tiers (0-35, 36-65, 66-85, 86+) and updated in real time.

In another embodiment, the system as described herein may include additional features such as rapid deployment and cost optimization. For example, implementations may complete in two to four weeks with high operational savings and no IT staff required.

In another embodiment, the system as described herein may include additional features such as a frictionless access method. In this configuration, evaluation respondents may participate without authentication or account creation, instead receiving session-based temporary identifiers that preserve anonymity while enabling data aggregation. The frictionless access method may further support multi-channel entry points, including unique URLs, QR codes, and mobile-optimized links, thereby maximizing response rates and reducing barriers to participation.

### Exemplary System for Monitoring and Analyzing Social Health for a Location

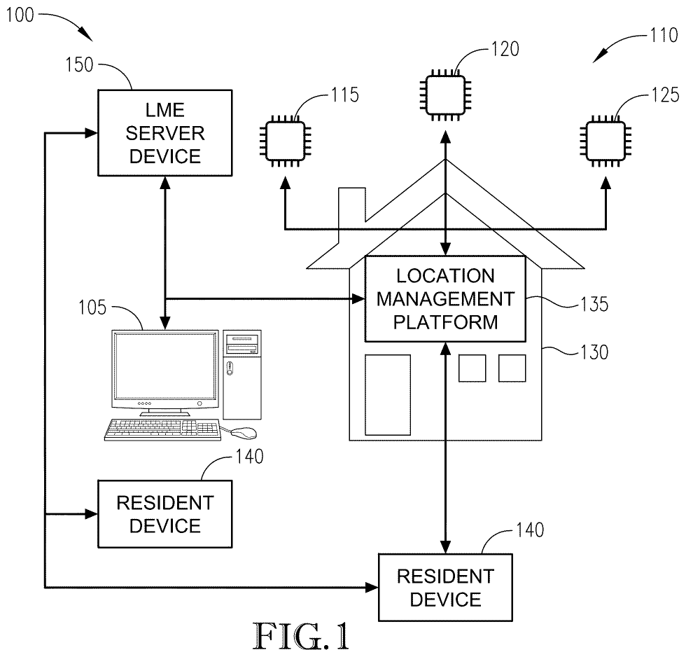

FIG. 1 illustrates an exemplary system 100 for monitoring and analyzing social heath factors of a location 130, in accordance with at least one embodiment of this disclosure. System 100 illustrates monitoring and other devices to receive, analyze, and report the data collected about the location 130.

In the exemplary embodiment, a location manager 105 provides one or more internet connected devices 110, also known as Internet of Things (IoT) devices 110. These devices 110 may be in or around a location 130. The devices 110 may include, but are not limited to IoT cameras 115, IoT thermostats 120, IoT door locks 125, and/or any other Internet connected device, such as a mobile device, including, but not limited to, a laptop and/or a mobile phone, one or more voice or chat bots, a computer device, including, but not limited to, a desktop computer and/or a router, and/or a location management platform 135. In at least one embodiment, the location management platform 135 is in wired or wireless communication the one or more devices 110 in the location 130. In some embodiments, the location management platform 135 may be a router or Wi-Fi providing device in the location 130. In other embodiments, the location management platform 135 is a smart location management platform that controls one or more of the devices 110 and may provide communication between the location manager and the resident devices 140.

In at least one embodiment, each device 110 collects data about the location 130, either directly or indirectly. For example, a smart light bulb may report when the bulb is on and off. This may indirectly indicate whether or not an individual is near the bulb. In at least one embodiment, many devices 110 are in communication with one or more servers of the location manager 105. The location manager server 105 may provide additional services, such as remote activation of one or more devices 110. The location manager server 105 may also collect data observed by the devices 110, including, but not limited to, usage data about the devices 110.

In some embodiments, a location monitoring and evaluation (LME) server 150 may be in communication with one or more of the IoT devices 110, the location management platform 135, one or more resident devices 140, and/or the location manager server 105. The LME server 150 collects data from the IoT devices 110 to determine the social health for the location 130. In various embodiments, the LME server 150 receives evaluation data from a plurality of resident devices 140 to determine the social health for the location 130. Then the LME server 150 determines one or more solutions and/or services that may correct and/or improve the social health for the location 130. The LME server 150 may utilize one or more AI/ML tools to generate the solutions and/or services. In various embodiments the solution may include an individualized solution for a resident associated with the resident evaluation data responses.

In various embodiments, the LME server 150 provides the evaluation to a resident device 140. The evaluation can also be administered physically and entered into the system 100. In various embodiments, the resident interacts with the resident device 140 to provide the resident data. In various embodiments, the system 100 and/or the LME server 150 generates the resident evaluation data questions. The resident evaluation data questions can be generated by a machine learning module. In various embodiments, the evaluation includes a plurality of resident evaluation questions based, at least in part, on the location data. The questions can be correlated to multiple social health factors. The social health factors can correspond to the social determinants of health (SDOH) outlined by government agencies. The resident evaluation can be generated to collect information from the residents based on a resident profile. For example, the evaluation may answer questions that indicate the social health of a resident of a location 130. The LME server 150 can process the evaluation data to identify social health factors associated with the resident and social health factors associated with the location 130. Accordingly, the resident evaluation data can be processed by the LME server 150 to generate the location social health score and the resident social health score, and the community social health score.

In the exemplary embodiment, resident devices 140 may also have sensors and other information that may be provided to the LME server 150 for analysis. For example, the LME server 150 may receive information from the resident device 140 through interaction with a resident device 140. From the resident device 140, the LME server 150 may receive information about the condition and social health of the resident through the resident device 140. The LME server 150 may then determine one or more social health factors associated with the resident device 140 and provide suggestions of one or more improvements and/or community services to improve the social health of the resident. The LME server 150 may receive information about the social health and living conditions of the resident through the interaction with the resident device 140 and determine that additional services would improve the social health of the resident. This may cause the LME server 150 to suggest that the resident associated with the resident device 140 receive additional services and/or provide information about resources so that the resident may control the types of assistance that they receive.

### Exemplary Location Monitoring System

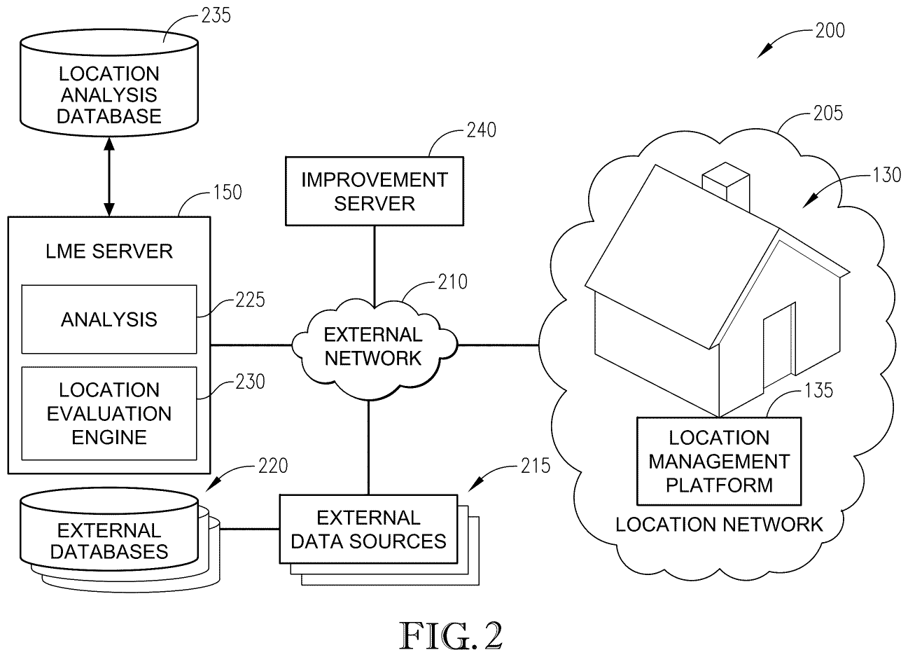

FIG. 2 illustrates an expanded location monitoring and evaluation (“LME”) system 200 that may be used for evaluating a location 130 and the factors associated with the location 130 and for providing solutions to improve those factors, in accordance with the present disclosure. In the exemplary embodiment, the LME system 200 includes the location monitoring and evaluation (LME) server 150 that may be remote from the location 130. The LME server 150 is configured to execute a location monitor and analysis engine 225 and a location evaluation engine 230. The LME server 150 may include or otherwise be in communication with a location analysis database 235 that stores information about the location 130 that may be used to evaluate location health and risks and may include information about real estate upon which the location 130 is located, residents associated within the location 130, and various data points that may influence the various factors of social health described herein. Collectively, this data is referred to herein as “community social health data.” Further, the terms “community,” “location,” and “property” may be used interchangeably herein to refer to the location 130 and its various residents and assets.

In the exemplary embodiment, the LME server 150 is in network communication with a location management platform (or just “platform”) 135 of the location 130 through an external network 210 (e.g., the Internet). The location management platform 135 may manage aspects of location social health data collection, computations, and alerting as a part of the system 100 (shown in FIG. 1). The location management platform 135 is connected to a location network 205 of the location 130 which allows communication with the LME server 150 through an external network 210 (e.g., the Internet). For example, the location network 205 may include a local area network (“LAN”), a wireless network (e.g., Wi-Fi network), or some combination thereof that connects to the external network 210 (e.g., via a subscription service to an Internet service provider, or the like). In some embodiments, the location management platform 135 may communicate via a wireless mobile network, such as a 3G, 4G, or 5G network.

In some embodiments, the systems 100 and 200 may be deployed on cloud computing infrastructure to provide scalability, reliability, and geographic distribution. For example, the LME server 150 may be hosted on a cloud computing platform utilizing containerized application deployment services (such as AWS ECS Fargate or similar container orchestration services) to enable elastic scaling of compute resources based on demand. Static content, such as images, stylesheets, and scripts, may be served via a content delivery network (CDN) to reduce latency and improve user experience for geographically distributed residents. Application credentials, API keys, and other sensitive configuration data may be stored in a secure credential management service (such as AWS Secrets Manager or similar secure key storage services) to prevent unauthorized access. The database infrastructure may employ NoSQL database systems optimized for scalability and flexible data models, enabling efficient storage and retrieval of hierarchical evaluation data, location data, and user account information.

In the exemplary embodiment, the systems 100 and 200 may allow location managers and residents to opt into or out of various aspects of data collection from location devices 110 (e.g., by device type, by type of data collected, by data use). For example, the resident and/or the location manager may be presented with an individual login to the system 100 and 200 which may include an opt-in screen that allows the location manager and/or resident to view data collection and usage policy and select whether they wish to allow such usage, thereby protecting privacy of the resident and/or location manager. Location data generated by such devices 110 may be referred to herein as just “location data.”

In some embodiments, the systems 100 and 200 may implement a user management system that defines distinct access levels for different types of users. For example, an administrator (“Admin”) account may be provided for internal users who configure customers, create and manage evaluations, and view aggregate analytics across multiple properties. A customer account, typically associated with landlords or property managers, may be configured to allow management of properties, assignment of evaluations to residents, and access to dashboards displaying property-level social health scores. A tenant account may be provided for anonymous evaluation respondents, wherein tenants access evaluations through unique URLs or QR codes without requiring login credentials, thereby preserving anonymity while still enabling collection of resident social health data. This tiered user management system ensures that each user type interacts only with the data and functionality appropriate to their role, thereby maintaining privacy and security while supporting efficient operation of the platform.

In some embodiments, the systems 100 and 200 may implement security and privacy protections for all communications between resident devices, the location management platform 135, and the LME server 150. For example, all data transmissions, including resident evaluation data and location data, may be protected using end-to-end encryption such that the data is encrypted on the transmitting device and decrypted only by the intended recipient, thereby preventing interception or unauthorized access during transit. In addition, each API (application programming interface) request between the resident devices and the LME server 150 may be authenticated using a JSON Web Token (JWT) stored in a secure cookie, ensuring that only authorized sessions can access system resources. For example, when a resident device submits evaluation responses, the responses are encrypted before transmission and accompanied by a JWT cookie identifying the authenticated session; the LME server 150 validates the token prior to accepting the encrypted payload, thereby enforcing both confidentiality and integrity of the resident evaluation data.

In some embodiments, the systems 100 and 200 may implement a secure user authentication flow for customer and administrator accounts. Upon account creation, the system may generate a temporary password and transmit it to the user's registered email address. When the user first logs in using the temporary password, the system may enforce a mandatory password reset, wherein the user is required to create a new, secure password before accessing platform functionality. In some embodiments, the system may further provide self-service password reset capabilities, wherein users who have forgotten their passwords can initiate a password reset process via email verification links. All user authentication actions, including logins, password resets, and failed authentication attempts, may be logged and associated with the specific user account for audit trail and security monitoring purposes.

In some embodiments, the systems 100 and 200 further implement secure API key handling to protect communications between the resident devices, the location management platform 135, and the LME server 150. For example, API keys used for accessing system resources may be stored only in secure server-side environments and are never exposed to client devices or embedded in application code. Each API request may include a server-validated key that is checked against a credential store before the request is processed, thereby ensuring that only authorized services can access protected endpoints. For example, when the location management platform 135 requests updated community social health scores from the LME server 150, the request is accompanied by a server-side API key that is validated against a secure credential manager; if the key is invalid or missing, the request is rejected. This approach enforces security best practices across development and deployment, reducing the risk of unauthorized access or credential leakage.

In some embodiments, the systems 100 and 200 are further configured to ensure complete anonymization of resident evaluation responses. For example, each evaluation response may be linked only to a property identifier and not to any individual resident or tenant, such that no personally identifiable information (PII) is stored or associated with the evaluation data. The system 100 may generate unique anonymous session identifiers for each evaluation instance, and the responses are aggregated at the property level before being processed by the LME server 150. As a result, landlords, location managers, and other stakeholders are provided only with anonymized, property-level analytics and community social health scores, thereby preserving resident privacy while still enabling meaningful evaluation of social health factors across different properties and communities.

In some embodiments, the systems 100 and 200 may implement granular permission scoping to control access to resident evaluation data, location data, and community social health scores based on user type. For example, an administrator account may be permitted to configure evaluations and view aggregate analytics across multiple properties, while a location manager account may be limited to viewing anonymized, property-level scores and recommended improvements for only those properties under their management. In contrast, a resident account may be restricted to completing evaluations and viewing only their own anonymized results without access to broader community data. This permission scoping ensures that each user type interacts only with the data necessary for their role, thereby maintaining privacy protections while still enabling effective use of the platform.

In some embodiments, the systems 100 and 200 are further configured to compute and display total response counts and overall score calculations for a given evaluation or property. For example, the LME server 150 may aggregate the number of completed resident evaluations associated with a location and calculate an overall community social health score based on the aggregated responses. The total response count may be presented alongside the calculated score to provide context regarding the statistical reliability of the results. In some embodiments, the system may further normalize the overall score to account for varying response rates across different properties, thereby ensuring that the community social health score reflects both the quality and quantity of resident participation.

In some embodiments, the systems 100 and 200 may provide evaluation data replay and filtering capabilities to ensure data quality and enable longitudinal analysis. For example, administrators may access historical evaluation response data and apply filtering rules to identify and exclude potentially malicious, incomplete, or anomalous responses that could distort aggregate social health scores. The system may implement automated or manual review processes wherein flagged responses are presented for administrator evaluation and selective exclusion from score calculations. In some embodiments, the system may further track community social health scores over time, generating temporal trend analyses that illustrate changes in resident well-being and social determinant factors across days, weeks, months, or years. These change-over-time tracking capabilities enable property managers and stakeholders to measure the impact of intervention programs, property improvements, or community services on overall social health outcomes.

In some embodiments, the systems 100 and 200 may also support granular data analysis at the property level. For example, the LME server 150 may generate property-specific dashboards that break down evaluation responses and social health scores by individual property, enabling landlords or location managers with multiple properties to compare performance across their portfolios. The system may allow filtering of results by property attributes, such as building type, size, or resident demographics, to identify localized trends and targeted opportunities for improvement.

In some embodiments, the systems 100 and 200 may support customer-specific dashboard customization to accommodate varying branding and data presentation preferences. For example, each customer account may be configured with custom branding elements, such as uploaded logo graphics that appear within the customer's dashboard interface, thereby reinforcing brand identity and user experience. The system may further provide configurable dashboard layouts wherein administrators or customers can select from predefined dashboard templates or customize data visualization components using drag-and-drop interface tools, enabling non-technical users to configure data displays without requiring programming expertise. In some embodiments, dashboard configurations may include drop-down selection menus for filtering data by property attributes, time periods, or social health categories, thereby facilitating intuitive navigation and analysis of community social health data.

In some embodiments, the systems 100 and 200 may employ a hierarchical data model architecture that organizes information across multiple levels of stakeholders and assets. At the highest level, a landlord entity may be associated with one or more properties. Each property may, in turn, be linked to one or more evaluations, and each evaluation may generate a plurality of resident responses. This hierarchical structure (Landlord→Properties→Evaluations→Responses) enables the system to maintain clear relationships between organizational entities, physical locations, and the data collected from residents, while supporting scalable management of large portfolios.

In some embodiments, the systems 100 and 200 may expose API endpoints to allow external systems, such as government entities or public health organizations, to securely access anonymized and aggregated evaluation data. These API endpoints may be configured with authentication mechanisms to ensure that only authorized entities, such as a city, a township, a county or similar organizations, can retrieve the data. In some embodiments, the system may also support integration with existing property management platforms, enabling landlords and property managers to synchronize property attributes, resident rosters, and evaluation assignments directly from their existing workflows into the community social health platform.

In some embodiments, the systems 100 and 200 may generate unique quick response (QR) codes for each evaluation instance to facilitate convenient evaluation distribution and resident access. Each QR code may encode a unique uniform resource locator (URL) corresponding to a specific property and evaluation combination, wherein the URL includes randomized anonymous identifiers to preserve resident privacy. When a resident scans the QR code using a mobile device, the resident device is automatically directed to the evaluation interface without requiring login credentials or personally identifiable information, thereby reducing barriers to participation while maintaining anonymity. Property managers may print or display the QR codes in common areas, include them in resident communications, or distribute them via digital channels, enabling flexible and accessible evaluation deployment across diverse resident populations.

In some embodiments, each property may have multiple evaluations enabled simultaneously, allowing landlords, location managers, property site, or location site to gather resident feedback on different social health factors or community initiatives in parallel. Each evaluation response may be assigned a unique anonymous session identifier generated by the system, ensuring that responses can be tracked and aggregated without linking them to personally identifiable information. This approach preserves resident privacy while still enabling longitudinal analysis of evaluation participation and outcomes.

In some embodiments, the systems 100 and 200 may implement multi-language support for resident evaluations to ensure accessibility across diverse resident populations. For example, each evaluation may be configured to display evaluation questions in multiple languages, such as English, Spanish, or French, with additional languages configurable as needed. In some embodiments, a language selection interface is presented to the resident prior to evaluation commencement, wherein the resident selects a preferred language from available options. Upon language selection, all evaluation questions, instructions, and user interface elements can be dynamically presented in the selected language, thereby ensuring that residents can comprehend and respond to evaluation questions in their native or preferred language. Each evaluation instance may generate unique URLs or QR codes corresponding to different language options, enabling property managers to distribute language-specific evaluation access points to resident populations as appropriate.

In some embodiments, the resident evaluation may employ a Likert scale response format for evaluation questions, wherein each question presents a range of response options from strongly disagree to strongly agree. The system may further enhance user experience by associating each response option with visual indicators, such as emoji-based graphics, to facilitate intuitive response selection by residents. In some embodiments, the evaluation platform may support multiple question types beyond Likert scale responses, including binary yes/no questions, free text responses, or multiple-choice selections, wherein each question type is selectable during evaluation configuration by administrators to optimize data collection for specific social health factors.

In some embodiments, the data model may further support custom property attributes and metadata, such as building type, number of units, or demographic indicators, which may be used to filter or contextualize evaluation results. Landlords may manage multiple properties within a single customer organization, and the system may support multiple users per customer organization, each with role-based access to the properties and evaluations under their purview. Properties may be linked to evaluations through explicit evaluation assignments, ensuring that only designated evaluations are active for a given property at a given time. This flexible and extensible data model architecture allows the system to accommodate diverse customer types and organizational structures while maintaining data integrity and privacy. The LME server 150, in the exemplary embodiment, may collect some location data from one or more external data sources 215. The location monitor and analysis engine 225 or the location evaluation engine 230 may, for example, collect data from publicly available sources or from private third-party sources about the particular subject location 130 or the area in which the location 130 is positioned (referred to herein as “the locality of the location”). For example, one external data source 215 may be the national weather service (“NWS”), a branch of the national oceanic and atmospheric administration (“NOAA”). The NWS collects, and makes publicly available, weather data for the United States of America and its outlying countries. Other external data sources may also be accessed and used.

The systems 100 and 200 may collect aspects of historical, current, or predictive weather data for a locality of the location 130 (e.g., community engagement, access to social services, inclusion, or the like) or from man-made challenges or other inherent social risks to a community (e.g., isolation due to lack of communal spaces, crime or lack of safety measures, inadequate social support systems or healthcare, education, or the like. Such data from external data sources 215 is referred to herein as “external location social health data,” or just “external data.” Some external data sources 215 may maintain such external data in one or more external databases 220. Other examples of external data sources 215 and external data may be provided by location management servers 105 (shown in FIG. 1) in addition to those provided below, as well as various uses for such external data.

In the exemplary embodiment, the LME server 150 is in communication with an improvement server 240 through the external network 210. The improvement server 240 is a platform where location managers and service providers coordinate improvements and provide services for the location. The improvement server 240 and the LME server 150 determine the needs of the users and then determines which location improvement providers and community service providers that may be of assistance to the resident. In various embodiments, the improvements correspond to an improvement in the social health score of the location.

In the exemplary embodiment, the LME server 150 may be operated by a location manager that is responsible for maintenance and improvements for the location 130 (e.g., via a location management contract) or that provides participation in systems 100 and 200 as a location improvement service for the location manager. The location manager may be any individual, group of individuals, company, corporation, or other type of entity that may provide improvements and services for locations, such as apartments, homes, or residential locations associated with the location 130. For example, after the location manager takes responsibility for the location, the location manager may provide the location management platform 135 for the location 130.

Although the present disclosure describes the systems and methods as being facilitated in part by the location manager, it should be appreciated that other non-location management related entities may implement the systems and methods. For example, a general contractor may aggregate the location data across many properties to determine which improvements or services provide the best improvement for specific social health factors, or deploy the improvements or services based upon where a decrease in social health is most likely to occur. Accordingly, it may not be necessary for the location 130 to have an associated location management contract for the residents of the location to enjoy the benefits of the systems and methods described herein.

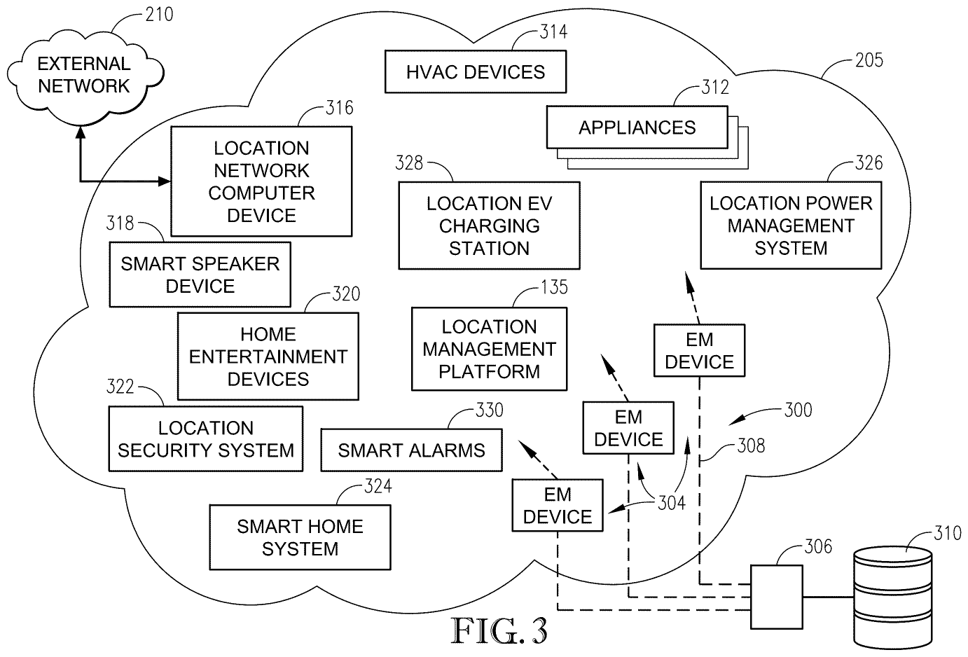

The location management platform 135, as discussed in greater detail below, may be configured to monitor aspects of location social health, collect location social health data from sensors, resident devices, or other devices within the location 130, connect to the location network 205, and communicate with the LME server 150 and/or improvement server 240 for the various aspects of location social health services and community social health evaluation described herein. The location management platform 135 may be configured to connect to the location network 205 and communicate with other networked devices 110 (or “smart devices”) within the location 130. Such devices 110 may be referred to herein as “source devices,” “connected devices,” “resident devices,” or “IoT devices,” as devices that provide location data to the systems 100 and 200. In some embodiments, the LME server 150 may communicate directly some or all of the source devices 110 within the location 130. Various resident devices 110 are illustrated in further detail below with respect to FIG. 3.

In the exemplary embodiment, the LME server 150 provides the users access to the improvement platform, while using ML (machine learning) and AI (artificial intelligence) to determine which location improvement service providers and community service providers are the most relevant to the user based on the analysis of the location and/or the residents social health. In at least some embodiments, the LME server 150 determines different attributes and/or conditions of the location based on the location data provided from the devices 110 and/or the external data sources 215. The LME server 150 may use the different attributes and/or conditions of the location to build a digital location profile of the location. Then the LME server 150 may use the digital location profile and the current location data to determine a location health score. The location health score is an abstraction that provides a value for the current condition of the location based on the aggregate of different attributes and/or conditions of the location. In the exemplary embodiment, the location health score is a mix of characteristics to provide a holistic view regarding the social health of a location. Elements about the location, as well as the surrounding environment, are taken into consideration as part of the calculation for the location social health score. The LME server 150 may also determine/calculate a community social health score that is an aggregate score displayed to the user, such as through a dashboard, which calculates subcategories of social health for a location based on location and external data. The location profile score may be provided through a mobile app that allows a user to search for their location and see a dynamic “Location Profile Dashboard” that includes a personalized Social Health Score, Recommendations, Improvements and Services.

In some embodiments, the LME server 150 may employ real-time predictive analytics, including the use of machine learning or artificial intelligence models, to anticipate changes in community social health conditions as new data is received. For example, as resident evaluation responses and location data are ingested by the LME server 150, one or more predictive models may continuously update forecasts of the community social health score, identifying emerging risks or opportunities before they are reflected in aggregate results. In some embodiments, the machine learning model may be trained on historical evaluation data, location data, and prior community outcomes to detect patterns indicative of declining or improving social health factors. The predictive analytics may generate early-warning indicators, such as detecting a downward trend in housing stability or social engagement and may trigger proactive recommendations or alerts to location managers. In some embodiments, the predictive analytics outputs may be displayed to users through a dashboard, which may include graphical visualizations such as trend lines, heat maps, or categorical breakdowns of predicted social health factors. This real-time, AI-driven forecasting capability, coupled with dashboard visualization, enables stakeholders to quickly interpret predictive insights and implement targeted improvements that mitigate negative outcomes and enhance overall community well-being.

### Exemplary External Data Sources

In the exemplary embodiment, and referring to FIG. 2, the system 200 may collect various types of external data from external data sources 215 that may be used, for example, for location social health evaluation, for social health scoring, for generating location social health improvement recommendations, or other various uses described herein. Some external data sources 215 may provide publicly available data, where other external data sources 215 may be private, third-party sources. External data sources 215 may include a location service provider that provides location management services to the location manager and various data available or otherwise collected by that location service provider. In some embodiments, the LME server 150 may be operated by the location service provider and the location social health database may include data private to the location service provider (e.g., resident data, service information, or other proprietary information).

In the exemplary embodiment, one example external data source 215 is the U.S. Department of Health and Human Services or any of its various branches (e.g., the office of disease prevention and health promotion). The HSS makes various health data publicly available. As such, the system 200 may collect social health data from the HSS. Such social health data may be refined to a particular geography, such as a state, county, city, or other geographic region. The system 200 may, for example, identify a geographic region of the location 130 and submit data queries to the HSS for health data specific to that geographic region. Such data queries may include requests for historical data such as these inquiries might cover historical statistics on factors such as community health outcomes, accessibility to healthcare services, environmental exposures, socioeconomic conditions, lifestyle choices, or the like. Historical data may be used to, for example, evaluate future improvements for the location 130 over time. Data queries may include requests for forecast data such as potential health risk factors, emerging health trends, or the like. Forecast data may be used to, for example, generate and send social health alerts to the location managers or residents of the location 130 or determine the susceptibility for the location 130 to experience various social health trends over time.

In the exemplary embodiment, another example external data source 215 may be third-party location data systems. The system 200 may access such location data systems to collect construction details about the location 130 such as, for example, the age of the location, amenities at the location 130, the type of residences at the location, the size of the location 130, market value of the location, whether the location 130 is constructed of wood, brick, concrete, or the like, or mobility or accessibility options within the location 130. Some location data may include geographic data about the location 130 such as, for example, school district information (e.g., public school system, school ratings), utility providers available to at the location (e.g., electric, gas, sewer, waste, recycling, phone, Internet, television, fire, police, hospital, or other city services), proximity data to various services and amenities (e.g., distances from schools, parks, grocery, gas, library, or sources of entertainment), and/or hazard data for the area (e.g., crime statistics, natural disaster statistics, ratings for emergency services). Some location statistics data may include historical data, such as social health history, public tax history, improvement history, community services information, location inspection information, lease information (e.g., whether and how often the location 130 has been partially or fully rented or leased), or the like. Some location statistics data may include location data such as, for example, whether the location 130 is energy certified, type and size of power generation, location appliance or lighting energy certification data, or the like.

In the exemplary embodiment, another example external data source 215 may be a location service provider or other service provider that has an economic or consumer relationship with the location. The system 200 may access the service provider systems to collect demographic details about the location 130 and its residents, such as, for example, names or ages of the residents, education levels or occupations of the occupants, whether any of the residents smoke, a resident emergency plan, social health of the residents, community engagement of the residents, or economic operations of the residents at the location 130. The service provider system may collect location maintenance data about the location 130 such as, for example, maintenance logs of operations performed on the location 130 (e.g., service calls, property damage and fixes, routine device maintenance, cleanings, bug or pest service, lawn or garden service, roofing replacement, social health service interactions, or the like), equipment installations and removals, device warranty information, or location improvements (e.g., new amenities, social health services, interior or exterior improvements or weather proofing, solar installation, water reclamation systems installation, room remodeling, or the like). The service provider system may collect location configuration data about the location 130 such as, for example, whether community social health services are provided at the location 130, whether social health services are supporting residents at the location 130. The system 100 may use external data to, for example, generate and send alerts to the location manager during or as factors in location social health scoring.

In some embodiments, the service provider may be the operator of the LME server 150, the residents and the location manager may provide such data via an input interface (e.g., online questionnaire, user interface, service application, or the like, during participation in the location health system described herein). Collection and use of such data may be opted into by the location manager on behalf of the residents. In various embodiments, the residents may opt out of resident data collection. In some embodiments, the system 200 may query the location manager for any data elements described herein and not otherwise automatically accessed by the system 200. The system 200 may generate a community health score for the location 130 using the external data, where the community social health score evaluates social health factors of the location 130 and the residents.

### Exemplary Resident Devices

FIG. 3 illustrates exemplary resident devices and systems that may be used with the system 100 (shown in FIG. 1) and the system 200 (shown in FIG. 2). In some embodiments, the location management platform 135 is in communication with, and/or otherwise monitors or collects data from, a variety of resident devices within the location communication system 300. The location 130 (shown in FIG. 1), and the various resident devices therein, may be connected by the location communication system 300, which is coordinated by a location communication platform 306 that can receive data from a location data source 310. Paths of communication flow are illustrated in FIG. 3 in broken lines. The multiple communication platforms 308 interface device-specific protocols with the location communication platform 306 so that heterogeneous resident devices can provide information to one or more devices and services within the location 130 and to the location management platform 135. The location communication platform 306 can receive data from at least one location data source 310, such as a resident evaluation response, a resident profile, a location profile, or an on-premises data source (e.g., local database, community database, social media), and forwards the data through the location communication system 300 toward the location management platform 135 for monitoring, analytics, and control.

In some embodiments, the location network computer device 316 operates as a local gateway, edge compute node, or controller within the location communication system 300. The Location Network Computer device 316 can exchange data and control messages with the location communication platform 306, aggregates telemetry from multiple communication platforms 308, and prepares device-normalized status updates for transmission to the location management platform 135 via the external networks 210. The device 316 may also cache configuration data and implement local failover logic, ensuring continued coordination of devices such as appliances 312, HVAC devices 314, home entertainment devices 320, smart speaker device 318, smart alarms 330, location security system 322, location EV charging station 328, and the location power management system 326 when connectivity to external networks 210 is intermittent.

Appliances 312 may be integrated into the location communication system 300 to report operational status (e.g., cycle state, fault codes) and energy consumption via the multiple communication platforms 308 to the location communication platform 306. HVAC devices 314 may provide environmental telemetry (e.g., temperature, humidity, airflow) and receive setpoints or schedules from the location management platform 135. Home entertainment devices 320 can share usage data and accept control commands (e.g., power, volume, input selection) through the location network computer device 316. The smart speaker device 318 enables voice-based resident interaction; voice commands and notifications are routed through the location communication platform 306 to the appropriate devices or to the location management platform 135. Smart alarms 330 (e.g., smoke, CO, water leak) can generate safety events and diagnostic signals; these are propagated by the multiple communication platforms 308 for immediate processing and potential alerting. The location security system 322 may include cameras, access control, motion sensors, and intrusion detection, and location security system 322 may exchange event data, video metadata, and control signals with the location communication platform 306 and, as configured, with the location management platform 135.

The location EV charging station 328 may report charging sessions, energy consumption, and availability, while receiving load-balancing or scheduling instructions from the location management platform 135; these exchanges can be brokered by the location network computer device 316 and the location communication platform 306. The location power management system 326 may coordinate energy usage across devices (e.g., HVAC devices 314, appliances 312, EV charging station 328, and home entertainment devices 320), using telemetry from EM device 304 and the multiple communication platforms 308 to dynamically optimize consumption and enforce demand-response policies. EM device 304 can provide energy or environmental monitoring data (e.g., real-time wattage, voltage, air quality metrics) to the location communication platform 306, which correlates such data with device telemetry and forwards aggregated insights to the location management platform 135 for analytics, alerts, and recommendations.

In some embodiments, the smart home system 324, as depicted in FIG. 3, represents the on-premises environment envelope that contains the aforementioned devices and subsystems. Smart home system 324 can coordinate communication between the location communication platform 306, the multiple communication platforms 308, the location network computer device 316, and the resident devices, and present a unified interface to the location management platform 135 over the external networks 210. Collectively, these components may operate within the location communication system 300 to ensure reliable data exchange, device interoperability, and centralized analytics and control. In some embodiments, the location management platform 135 ingests data from the location data source 310 and device telemetry via the location communication platform 306, computes insights or control strategies, and returns configuration updates or commands through the external networks 210 and the location network computer device 316 to the appropriate resident devices.

### Exemplary System

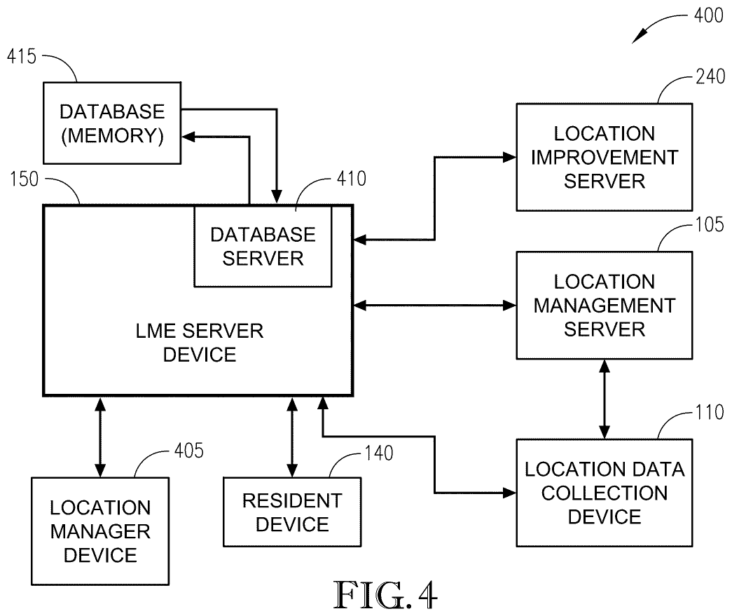

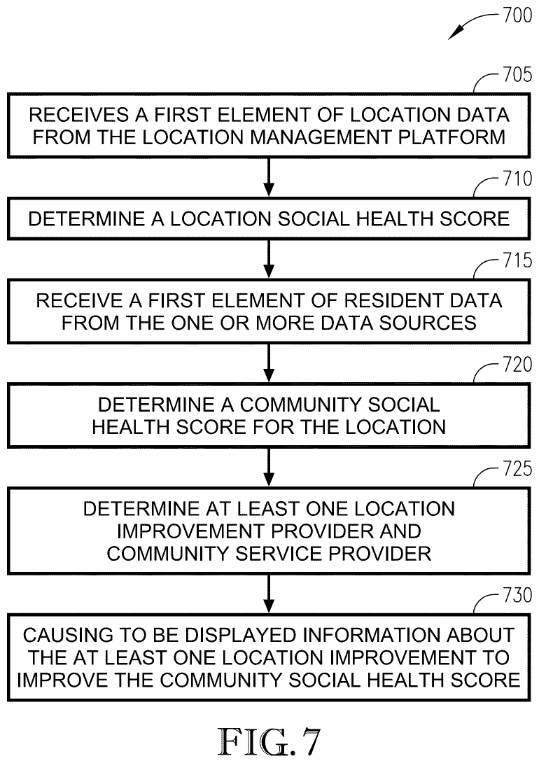

FIG. 4 illustrates an exemplary computer system 400 for implementing the systems 100 and 200 (shown in FIGS. 1 and 2) performing process 700 (shown in FIG. 7). In the example embodiment, the system 400 is used for analyzing resident data, sensor data, and external data associated with a location 130 (shown in FIG. 1) to detect social health conditions affecting the location and propose solutions to improve the social health factors of the location.

As described below in more detail, the location monitoring and evaluation (LME) server 150 is programmed to analyze resident data, sensor data, and external data associated with a location to detect issues with the social health of the location and to propose improvements to mitigate those issues. The LME server 150 is programmed to (1) receive a first element of home data from the location management platform 135 (shown in FIG. 1); (2) determine a location social health score for the location 130 based at least in part on the first element of location data, wherein the location social health score represents a measure of social health of the location 130; (3) receive a first element of external data from the one or more external data sources 215 (shown in FIG. 1), wherein the first element of external data relates to a resident of the location; (4) determine a community social health score for the location based at least in part on one or more of the first element of location data provided by the one or more resident devices 140 and the first element of external data from the one or more external data sources 215, wherein the community social health score represents a measure of social health of the community associated with the location 130; (5) determine at least one location improvement service improvement and community service provider to be able to improve the community social health score based at least in part upon the first element of location data and the first element of external data; and (6) cause to be displayed, to a location manager of the location 130 via a graphical user interface, information about the at least one location improvement service improvement and community service provider to improve the community health score.

In the example embodiment, location manager devices 405 are computers that include a web browser or a software application, which enables resident devices 140 to communicate with LME server 150 using the Internet, a local area network (LAN), or a wide area network (WAN). In some embodiments, the location manager devices 405 are communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem. Location manager devices 405 can be any device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chat bots, voice bots, ChatGPT bots or ChatGPT-based bots, or other web-based connectable equipment or mobile devices.

In the example embodiment, resident devices 140 are computers that include a web browser or a software application, which enables resident devices 140 to communicate with LME server 150 using the Internet, a local area network (LAN), or a wide area network (WAN). In some embodiments, the resident devices 140 are communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem. Resident devices 140 can be a device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chat bots, voice bots, ChatGPT bots or ChatGPT-based bots, or other web-based connectable equipment or mobile devices. In the exemplary embodiment, resident devices 140 as devices connected to the location network 205 (shown in FIG. 2) that provide information about the home/location 130.

In the example embodiment, location management servers 105 are computers that include a web browser or a software application, which enables location management servers 105 to communicate with associated resident devices 140 and the LME server 150 using the Internet, a local area network (LAN), or a wide area network (WAN). In some embodiments, the location management servers 105 are communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem. The location management servers 105 can be a device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chat bots, voice bots, ChatGPT bots or ChatGPT-based bots, or other web-based connectable equipment or mobile devices.

In the example embodiment, improvement servers 240 are computers that include a web browser or a software application, which enables improvement servers 240 to communicate with associated the LME server 150 using the Internet, a local area network (LAN), or a wide area network (WAN). In some embodiments, the improvement servers 240 are communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem. The improvement servers 240 can be a device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chat bots, voice bots, ChatGPT bots or ChatGPT-based bots, or other web-based connectable equipment or mobile devices.

In the example embodiment, the LME server 150 (also known as LME computer device 150) is a computer that includes a web browser or a software application, which enables LME server 150 to communicate with resident devices 805 using the Internet, a local area network (LAN), or a wide area network (WAN). In some embodiments, the LME server 150 is communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem. The LME server 150 can be a device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chat bots, voice bots, ChatGPT bots or ChatGPT-based bots, or other web-based connectable equipment or mobile devices.

A database server 410 is communicatively coupled to a database 415 that stores data. In one embodiment, the database 415 is a database that includes location data, resident data, sensor data, community data, and/or social health improvement actions. In some embodiments, the database 415 is stored remotely from the LME server 150. In some embodiments, the database 415 is decentralized. In the example embodiment, a person can access the database 415 via the location manager devices 405 by logging onto the LME server 150.

### Exemplary Location Manager Device

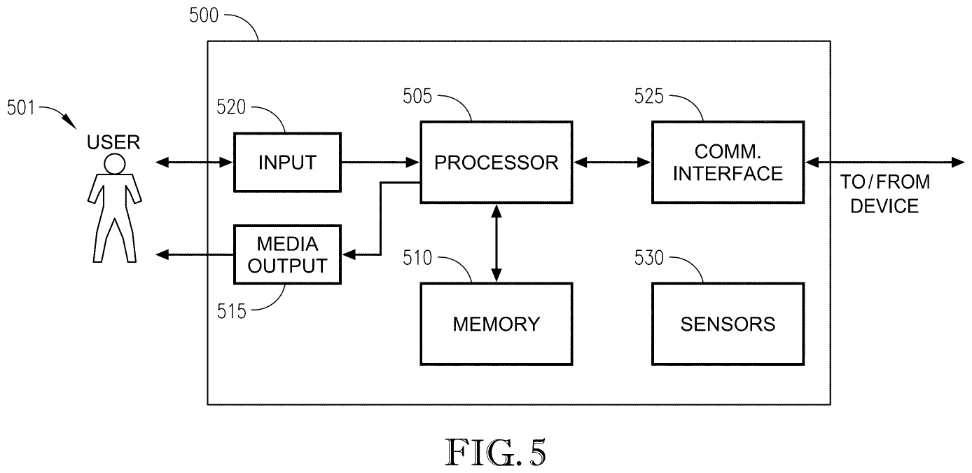

FIG. 5 depicts an exemplary configuration of a user computer device 500, in accordance with one embodiment of the present disclosure. User computer device 500 may be operated by a resident 501. User computer device 500 may include, but is not limited to, devices 110, IoT camera 115, IoT thermostat 120, IoT door lock 125, resident device 145 (all shown in FIG. 1), EM devices 304, HVAC devices 314, location network computer devices 316, smart speaker devices 318, home entertainment devices 320, home security system 322, smart home system 324, location power management system 326, location EV charging station 328 (all shown in FIG. 3), and location manager devices 405 (shown in FIG. 4). User computer device 500 may include a processor 505 for executing instructions. In some embodiments, executable instructions are stored in a memory area 510. Processor 505 may include one or more processing units (e.g., in a multi-core configuration). Memory area 510 may be any device allowing information such as executable instructions and/or transaction data to be stored and retrieved. Memory area 510 may include one or more computer readable media.

User computer device 500 may also include at least one media output component 515 for presenting information to resident 501. Media output component 515 may be any component capable of conveying information to resident 501. In some embodiments, media output component 515 may include an output adapter (not shown) such as a video adapter and/or an audio adapter. An output adapter may be operatively coupled to processor 505 and operatively connected to an output device such as a display device (e.g., a cathode ray tube (CRT), liquid crystal display (LCD), light emitting diode (LED) display, or “electronic ink” display), an audio output device (e.g., a speaker or headphones), virtual headsets (e.g., AR (Augmented Reality), VR (Virtual Reality), or XR (extended Reality) headsets), and/or voice or chat bots.

In some embodiments, media output component 515 may be configured to present a graphical user interface (e.g., a web browser and/or a client application) to resident 501. A graphical user interface may include, for example, an online score viewing interface for viewing a location social health score, resident social health score, or community social health score and determining more information about the score. In some embodiments, user computer device 500 may include an input device 520 for receiving input from resident 501. Resident 501 may use input device 520 to, without limitation, perform a resident evaluation.

Input device 520 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 515 and input device 520.

User computer device 500 may also include a communication interface 525, communicatively coupled to a remote device such as the LME server 150 (shown in FIG. 1) and/or the improvement server 240 (shown in FIG. 2). Communication interface 525 may include, for example, a wired or wireless network adapter and/or a wireless data transceiver for use with a mobile telecommunications network.

Stored in memory area 510 are, for example, computer readable instructions for providing a user interface to resident 501 via media output component 515 and, optionally, receiving and processing input from the resident device 520. A user interface may include, among other possibilities, a web browser and/or a client application. Web browsers enable users, such as resident 501, to display and interact with media and other information typically embedded on a web page or a website from the LME server 150 and/or the improvement server 240. A resident application allows resident 501 to interact with, for example, the LME server 150 and/or the improvement server 240. For example, instructions may be stored by a cloud service, and the output of the execution of the instructions sent to the media output component 515.

Processor 505 executes computer-executable instructions for implementing aspects of the disclosure. In some embodiments, the processor 505 is transformed into a special purpose microprocessor by executing computer-executable instructions or by otherwise being programmed.

User computer device 500 may further be operatively connected to one or more sensors 530. Sensors 530 may include, without limitation, motion sensors, temperature sensors, humidity sensors, light sensors, occupancy detectors, biometric sensors, or environmental monitoring sensors (e.g., air quality, CO₂, smoke, or noise level). In some embodiments, sensors 530 provide raw or pre-processed data streams to processor 505 via communication interface 525, where the data may be stored in memory area 510 and analyzed locally or transmitted to the LME server 150 and/or improvement server 240. The information collected by sensors 530 may be used to augment resident evaluation responses, validate environmental conditions reported by HVAC devices 314 or EM devices 304, and trigger automated actions through the location management platform 135. For example, occupancy data from sensors 530 may be combined with input from smart speaker devices 318 or home security system 322 to adjust HVAC settings, activate smart alarms 330, or generate alerts to resident 501 through media output component 515. In this way, sensors 530 extend the functionality of user computer device 500 by providing continuous, context-aware data that enhances both resident experience and system intelligence.

### Exemplary Server Device

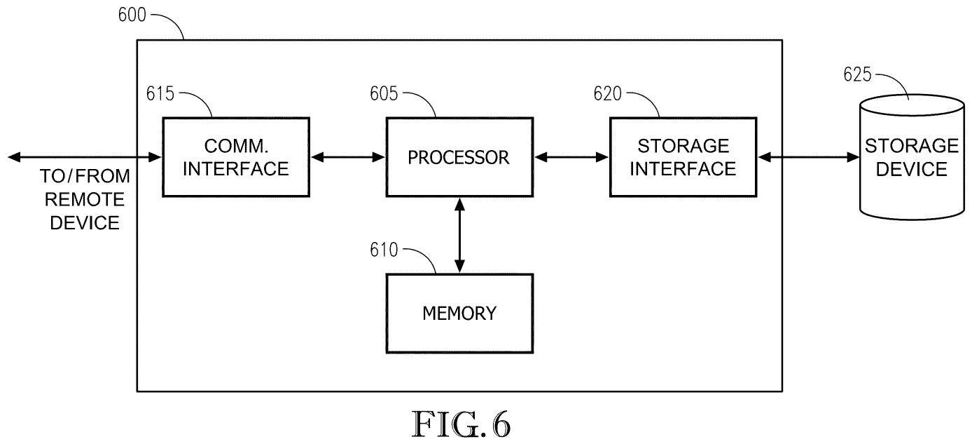

FIG. 6 depicts an exemplary configuration of a server computer device 600, in accordance with one embodiment of the present disclosure. Server computer device 600 may include, but is not limited to, location manager server 105, location management platform 135, LME server 150 (both shown in FIG. 1), external data sources 215, and improvement server 240 (both shown in FIG. 2). Location security system 322, smart home system 324, location power management system 326, (all shown in FIG. 3), and database server 410 (shown in FIG. 4) may be in communication with server computer device 600. Server computer device 600 may also include a processor 605 for executing instructions. Instructions may be stored in a memory area 610. Processor 605 may include one or more processing units (e.g., in a multi-core configuration).

Processor 605 may be operatively coupled to a communication interface 615 such that server computer device 600 is capable of communicating with a remote device such as another server computer device 600, improvement server 240, or location manager devices 405 (shown in FIG. 4). For example, communication interface 615 may receive requests from location manager devices 405 via the Internet, as illustrated in FIG. 4.

Processor 605 may also be operatively coupled to a storage device 625. Storage device 625 may be any computer-operated hardware suitable for storing and/or retrieving data, such as, but not limited to, data associated with database 415 (shown in FIG. 4). In some embodiments, storage device 625 may be integrated in server computer device 600. For example, server computer device 600 may include one or more hard disk drives as storage device 625.

In other embodiments, storage device 625 may be external to server computer device 600 and may be accessed by a plurality of server computer devices 600. For example, storage device 625 may include a storage area network (SAN), a network attached storage (NAS) system, and/or multiple storage units such as hard disks and/or solid-state disks in a redundant array of inexpensive disks (RAID) configuration.

In some embodiments, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Processor 605 may execute computer-executable instructions for implementing aspects of the disclosure. In some embodiments, the processor 605 may be transformed into a special purpose microprocessor by executing computer-executable instructions or by otherwise being programmed. For example, the processor 605 may be programmed with the instructions such as illustrated in FIG. 7.

### Exemplary Computer-Implemented Method for Monitoring and Updating Social Health

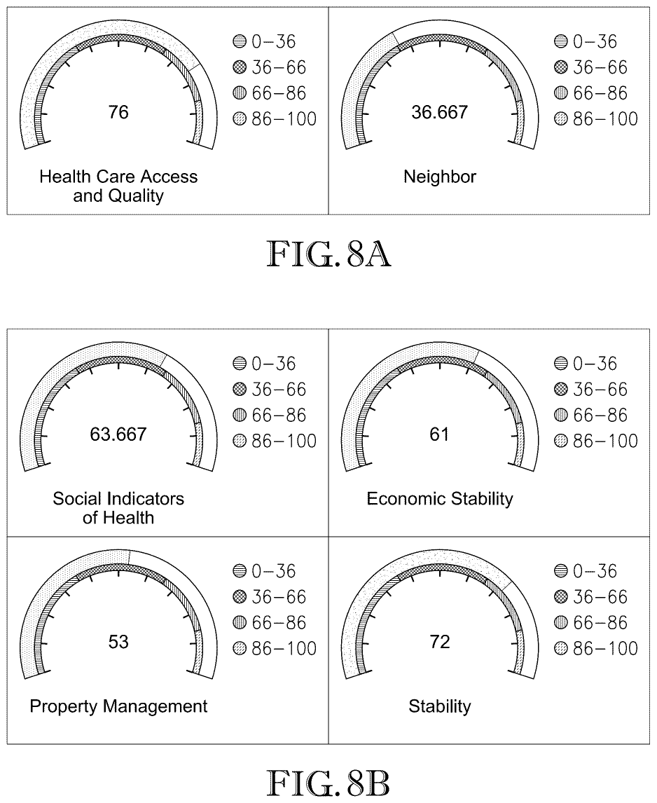

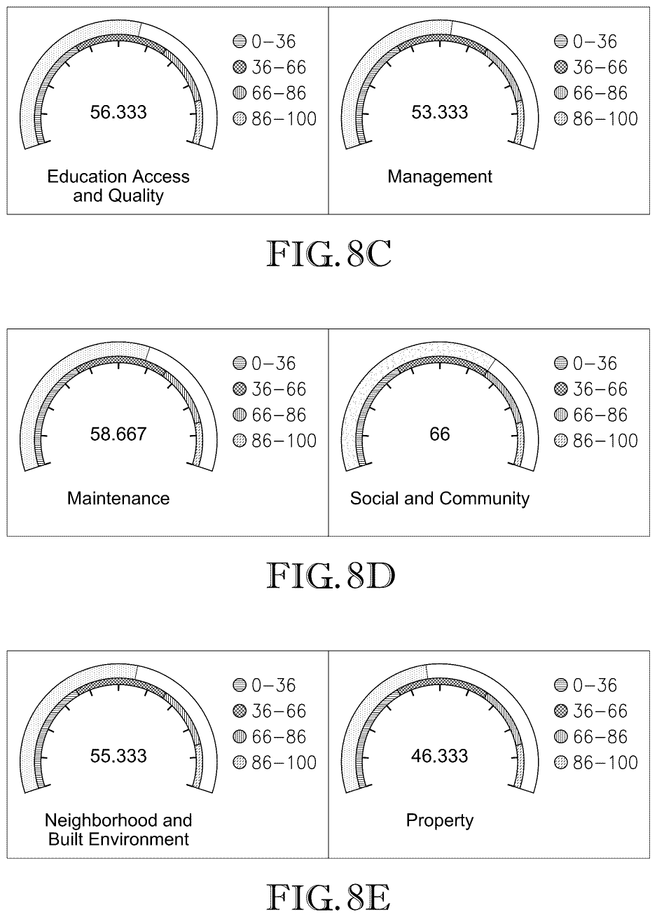

FIG. 7 depicts a flow chart of an exemplary computer-implemented process 700 for evaluating and improving social health of a location 130 (shown in FIG. 1) using the systems 100 and 200 (shown in FIGS. 1 and 2). Process 700 may be implemented by a computing device, for example LME server 150 (shown in FIG. 1). In the exemplary embodiment, LME server 150 may be in communication with one or more smart devices 110 installed within the location 130, one or more location management platforms 135 (all shown in FIG. 1), one or more external data sources 215, one or more improvement servers 240 (both shown in FIG. 2), and one or more resident devices 140 (shown in FIG. 8).

In the exemplary embodiment, the LME server 150 receives 705 a first element of location data from the location management platform 135. The location management platform may receive the first element of location data from one or more devices 110 on the location network 205 (shown in FIG. 2).

In the exemplary embodiment, the LME server 150 determines 710 a location social health score for the location 130 based at least in part on the first element of location data. The location social health score represents a measure of safety of the location 130.

In the exemplary embodiment, the LME server 150 receives 715 a first element of resident data from the one or more external data sources 215. The first element of external data relating to resident evaluation data corresponding to the location 130.

In the exemplary embodiment, the LME server 150 determines 720 a community health score for the location 130 based at least in part on one or more of the first element of location data provided by the one or more smart devices 110 and the first element of external data from the one or more external data sources 215. The community social health score represents a measure of health of the location and the community of residents associated with the location 130.

In the exemplary embodiment, the LME server 150 determines 725 at least one location improvement service provider and community service provider (such as shown in FIG. 2) to be able to improve the community health score based at least in part upon the first element of location data and the first element of external data.

In the exemplary embodiment, the LME server 150 causes 730 to be displayed, to a location manager of the location 130 via a graphical user interface, information about the at least one location improvement provider and community service provider to improve the community social health score.

FIGS. 8A-8E illustrate graphs of different scores in different categories based upon the systems and methods described herein.

FIG. 8A illustrates exemplary scoring outputs for categories such as Health Care Access and Quality and Neighbor. The graphs demonstrate how the system aggregates resident evaluation data into normalized scores, enabling comparison across multiple social determinant categories and highlighting areas requiring targeted improvements.

FIG. 8B illustrates exemplary scoring outputs for categories including Economic Stability, Social Indicators of Health, Property Management, and Stability. The graphs demonstrate how the system aggregates resident evaluation data into normalized scores, enabling comparison across multiple social determinant categories and highlighting areas requiring targeted improvements.

FIG. 8C illustrates exemplary scoring outputs for Education Access and Quality and Management. The graphs demonstrate how the system aggregates resident evaluation data into normalized scores, enabling comparison across multiple social determinant categories and highlighting areas requiring targeted improvements.

FIG. 8D illustrates exemplary scoring outputs for Maintenance and Social and Community. The graphs demonstrate how the system aggregates resident evaluation data into normalized scores, enabling comparison across multiple social determinant categories and highlighting areas requiring targeted improvements.

FIG. 8E illustrates exemplary scoring outputs for Neighborhood and Built Environment and Property. The graphs demonstrate how the system aggregates resident evaluation data into normalized scores, enabling comparison across multiple social determinant categories and highlighting areas requiring targeted improvements.

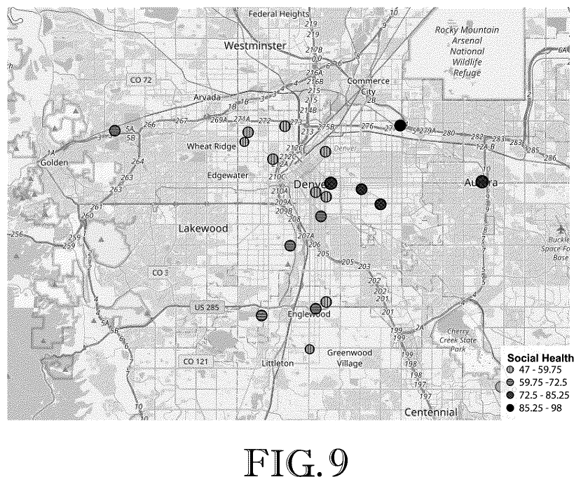

FIG. 9 illustrates a map of different residents and/or facilities and their corresponding categories, in accordance with at least one embodiment. The map visually represents geographic regions, neighborhoods, or facilities overlaid with social health scoring data generated by the system. Each location may be associated with one or more categories of social determinants of health, such as economic stability, education access, healthcare access, or neighborhood environment. The system may display these categories with corresponding scores or ranges, enabling stakeholders to quickly identify geographic disparities, high-risk areas, or facilities requiring targeted interventions. In some embodiments, the map may be interactive, allowing a user to select a region or facility to view detailed resident evaluation data, location data, and recommended improvements for increasing the community social health score.

### Machine Learning and Other Matters

The computer-implemented methods discussed herein may include additional, less, or alternate actions, including those discussed elsewhere herein. The methods may be implemented via one or more local or remote processors, transceivers, servers, and/or sensors (such as processors, transceivers, servers, and/or sensors mounted mobile devices, or associated with smart infrastructure or remote servers), and/or via computer-executable instructions stored on non-transitory computer-readable media or medium.

In some embodiments, LME computer system 150 is configured to implement machine learning, such that LME computer system 150 “learns” to analyze, organize, and/or process data without being explicitly programmed. Machine learning may be implemented through machine learning methods and algorithms (“ML methods and algorithms”). In an exemplary embodiment, a machine learning module (“ML module”) is configured to implement ML methods and algorithms. In some embodiments, ML methods and algorithms are applied to data inputs and generate machine learning outputs (“ML outputs”). Data inputs may include but are not limited to images and resident data. ML outputs may include, but are not limited to identified social health factors, item classifications, and/or other data extracted from the inputs. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, the ML module employs supervised learning, which involves identifying patterns in existing data to make predictions about subsequently received data. Specifically, the ML module is “trained” using training data, which includes example inputs and associated example outputs. Based upon the training data, the ML module may generate a predictive function which maps outputs to inputs and may utilize the predictive function to generate ML outputs based upon data inputs. The example inputs and example outputs of the training data may include any of the data inputs or ML outputs described above. In the exemplary embodiment, a processing element may be trained by providing it with a large sample of home attributes with known characteristics or features. Such information may include, for example, information associated with a plurality of devices 110.

In another embodiment, a ML module may employ unsupervised learning, which involves finding meaningful relationships in unorganized data. Unlike supervised learning, unsupervised learning does not involve user-initiated training based upon example inputs with associated outputs. Rather, in unsupervised learning, the ML module may organize unlabeled data according to a relationship determined by at least one ML method/algorithm employed by the ML module. Unorganized data may include any combination of data inputs and/or ML outputs as described above.

In yet another embodiment, a ML module may employ reinforcement learning, which involves optimizing outputs based upon feedback from a reward signal. Specifically, the ML module may receive a user-defined reward signal definition, receive a data input, utilize a decision-making model to generate a ML output based upon the data input, receive a reward signal based upon the reward signal definition and the ML output, and alter the decision-making model so as to receive a stronger reward signal for subsequently generated ML outputs. Other types of machine learning may also be employed, including deep or combined learning techniques.

In some embodiments, generative artificial intelligence (AI) models (also referred to as generative machine learning (ML) models) may be utilized with the present embodiments, and may the voice bots or chatbots discussed herein may be configured to utilize artificial intelligence and/or machine learning techniques. For instance, the voice or chatbot may be a ChatGPT chatbot. The chatbot may also use alternative Large Language Models (LLMs). The voice or chatbot may employ supervised or unsupervised machine learning techniques, which may be followed by, and/or used in conjunction with, reinforced or reinforcement learning techniques. The voice or chatbot may employ the techniques utilized for ChatGPT. The voice bot, chatbot, ChatGPT-based bot, ChatGPT bot, and/or other bots may generate audible or verbal output, text or textual output, visual or graphical output, output for use with speakers and/or display screens, and/or other types of output for user and/or other computer or bot consumption.

Based upon these analyses, the processing element may learn how to identify characteristics and patterns that may then be applied to analyzing and classifying social health data. The processing element may also learn how to identify attributes of social health data in different locations. This information may be used to determine which classification models to use and which classifications to provide.

### Exemplary Embodiments

In one aspect, a computer system may be provided. The computer system may include one or more local or remote processors, servers, sensors, memory units, transceivers, mobile devices, wearables, smart watches, smart glasses or contacts, augmented reality glasses, virtual reality headsets, mixed or extended reality headsets, voice bots, chat bots, ChatGPT bots, and/or other electronic or electrical components, which may be in wired or wireless communication with one another. For instance, the computer system may include at least one processor in communication with at least one memory device.

The at least one processor may be configured to: (i) receive a first element of location data from the location management platform; (ii) determine a location social health score for the location based at least in part on the first element of location data, wherein the location social health score represents a measure of social health of the location; (iii) receive a first element of external data from the one or more external data sources, wherein the first element of external data relates to a resident of the location; (iv) determine a community social health score for the location based at least in part on one or more of the first element of location data provided by the one or more devices and the first element of external data from the one or more external data sources, wherein the community social health score represents a measure of community social health of the location; (v) determine at least one location improvement recommendation and community service provider to be able to improve the community social health score based at least in part upon the first element of location data and the first element of external data; and/or (vi) cause to be displayed, to a location manager of the location via a graphical user interface, information about the at least one location improvement recommendation and service provider to improve the home health score. The system may have additional, less, or alternate functionality, including that discussed elsewhere herein.

In yet another aspect, a computer-implemented method of evaluating and improving aspects of a community social health score may be provided. The computer-implemented method may be performed by a computing device including at least one processor and/or associated transceiver. The method may include, via the at least one processor and/or associated transceiver: (i) receiving a first element of location data captured by one or more resident devices associated with the location, wherein the first element of location data reflects an aspect of social health of one or more assets of the location; (ii) determining a location social health score for the location based at least in part on the first element of location data, wherein the location social health score represents a measure of social health of the location; (iii) receiving a second element of location data captured by the one or more resident devices; (iv) determining a community social health score for the location based at least in part on one or more of the first element of location data and the second element of location data, wherein the community social health score represents a measure of social health of the location; (v) determining at least one location improvement recommendation and community service provider to be able to improve the community social health score based at least in part upon the first element of location data and the first element of external data; and/or (vi) causing to be displayed, to a location manager of the location via a graphical user interface, information about the at least one location improvement recommendation and community service provider to improve the community health score. The method may have additional, less, or alternate actions, including that discussed elsewhere herein.

In still another aspect, a non-transitory computer readable medium having computer-executable instructions embodied thereon for evaluating aspects of social health of a location may be provided. When executed by at least one processor and/or associated transceiver, the computer-executable instructions cause the at least one processor and/or associated transceiver to: (i) receive a first element of location data from the location management platform; (ii) determine a location social health score for the location based at least in part on the first element of location data, wherein the safety score represents a measure of social health of the location; (iii) receive a first element of external data from the one or more external data sources, wherein the first element of external data relates to a resident of the location; (iv) determine a community health score for the location based at least in part on one or more of the first element of location data provided by the one or more location management platforms and the first element of external data from the one or more external data sources, wherein the community social health score represents a measure of social health of the community associated with the location; (v) determine at least one location improvement recommendation and community service provider to be able to improve the community social health score based at least in part upon the first element of home data and the first element of external data; and/or (vi) cause to be displayed, to a location manager of the location via a graphical user interface, information about the at least one location improvement recommendation and community service provider to improve the community social health score. The computer readable medium may have instructions that direct additional, less, or alternate functionality, including that discussed elsewhere herein.

At least one advantage of the system described herein includes the ability to address social health issues of a location. The system provides an objective and repeatable process to assess social determinants of health through resident evaluations, processing location data, and identifying location improvements provided by location improvement providers and community service providers. The system generates a location social health score and a community social health score. The location social health score corresponds to the impact of the location on social health factors. The community social health score corresponds to the overall social health of a location and its corresponding community. The identified improvements correspond to solutions for location managers to improve the social determinants of health that impact the community social health score. The platform processes data from the location, residents, external databases, and community to compute a community social health score. The community social health score quantifies the effect of the location and resident social health on the community. For example, the community social health score can be impacted by location management operations, resident services, and community development initiatives. By processing resident evaluation data, location data, and external databases, the platform generates solutions to improve the community social health score of a location data. The platform can further assist in the identification, evaluation, and generation of intervention strategies to maximize both the location social health score and the community social health score.

The system processes the location data to quantify the social determinants of health impacting a location to generate a location social health score. By quantifying the social determinants of health into a social health score, the system can generate data driven solutions to maximize location management strategies. The system also generates a community social health score. For example, the generation of a community social health score provides data-driven improvements for location managers to increase the social health of a location. In various embodiments, the improvement to the location corresponds to providing additional community services for the residents. The additional community services correspond to the social determinants of health that contribute to the community social health score. For example, community services for the residents may include tenant services, location maintenance, and community development initiatives to increase the location social health. Additionally, the platform's location management systems allow for the implementation of the solutions to maximize the social health factors of a location. Accordingly, the system improves location management through data-driven solutions to identify the social health of a location and generating a community social health score to identify improvements benefitting the residents of the community associated with the location.

The system provides community social health report generation and analysis. The system generates improvements for the location to improve the social health of a location by addressing the social health factors, such as the social determinants of health (SDOH). Social determinants of health include social conditions and structural conditions. The SDOH may include healthcare access and quality, education access and quality, social and community context, economic stability, and neighborhood and built environment. Factors that contribute to the SDOH may include safe housing, transportation, and neighborhoods; polluted air and water; and access to nutritious foods and physical health opportunities. Accordingly, the improvements increasing the social health of the location thereby improving the community social health score of the location. In various embodiments, the system identifies improvements such as social investments based on the generates social health score. The system identifies factors of the SDOH and the location to generate the improvements. The resident evaluation data is then provided to the system to process the resident evaluation data to a correlation of a at least one SDOH factor.

To generate the social health report, the report generation process may associate each evaluation question with at least one social determinant of health factor. The social health report can include the social determinants of health that are measured by the generated evaluation questions. The report may process the weighted evaluation questions to generate a report for the plurality of social health factors. The question may be weighted to a first SDOH factor and a second SDOH factor. For example, a question about access to healthy food options could be correlated to a first SDOH factor and at least one additional SDOH factor. The platform may also assign weights to specific evaluation questions based on their correlation with multiple SDOH factors. The additional factors may be associated with additional weights.

Once the evaluation data has been linked to SDOH factors and weighted appropriately, the platform generates a social health score for each resident, location, and/or community. The platform computes the score to reflect the overall level of well-being in terms of the identified SDOH factors. The score may correspond to a range that reflects the overall status of the resident. For example, the score may fall within a range of one to one hundred. This range can be divided into categories, such as success, progressive, developing, and passable. The size of these categories may vary, or they may be evenly distributed. Based on this information, the platform generates data-driven improvements to provide social reinvestment strategies for the location. For example, if the platform detects poor access to healthcare services from the data extracted from the evaluation, the platform could access a community service provider to connect residents with local clinics or implement health screenings and education programs at the location.

The platform identifies solutions to increase the community social health score for a location. In various embodiments, the solution may include targeted interventions for the location. The platform identifies specific interventions or programs aimed at addressing the most impactful social health needs of the location. The social health needs can be based on the evaluation data, the location data, and the associated SDOH factors. For example, if access to education is a major concern identified from the evaluation results, the system identifies community initiatives and services to provide after-school tutoring or partnering with local schools to improve literacy rates. Accordingly, the solutions can thereby increase social health factors of a community such that it would improve the resulting social health score.

In various embodiments, the platform may access external databases to identify solutions. For example, analyzing trends in the evaluation data over time allows the system to identify long-term solutions that will impact location social health and improve residents' well-being. For example, when the community social health score indicates transportation concerns, the system may generate and identify public transportation infrastructure improvements. The system may identify the underlying social health issues to improve accessibility and the social health score for the location. In various embodiments, the platform generates maintenance, development, and upkeep programs for the location to maximize the social health improvement for the location based on the resident evaluation data. For example, the system can detect and track maintenance conditions that may affect the location social health score.

In various embodiments, the platform identifies residents that are indicating a decrease in their social health score. The platform may extract information from the resident evaluation data to detect the change in social health of the resident. The system monitors the social health of the resident over time to predict when external intervention is beneficial for the social health of the resident. For example, the platform may identify a resident that requires additional assistance based on their monitored social health score. The platform may connect the resident to additional assistance such as government programs or nonprofits corresponding to the change in the social health of the resident.

In the process of evaluating a property's social determinants of health (SDOH) impact, the platform may consider data surrounding the location. The surrounding area assessment involves collecting information on various aspects that can influence the SDOH factors of the residents living near the location. For example, the platform may utilize a plurality of public databases to supplement the evaluation data when computing the social impact score for a location. Referencing the community data provided a contextualization to the location contributing to the calculation for the social impact.

The system references community data through a combination of methods, including external databases, observation data, and evaluation data. The system leverages external databases including public records, demographic data, crime statistics, and other available information to gain insights into the broader context surrounding the social health of the location. In some embodiments, the system references data including socioeconomic indicators like median household income, poverty rates, educational attainment levels, and employment statistics when calculating the social health score for a location.

The system can process data collected from the location. In various embodiments, the location includes data collection devices such as cameras, sensors, and other observation devices located at the location to collect location data. The location data can be collected and processed to indicate the physical environment, infrastructure, and amenities associated with a location. In various embodiments, the system provides an evaluation to the residents of the community around the location. The system processes the community evaluation data to gain a deeper understanding of the community social health factors for the location. Based on the results from the system, the platform can identify community programs designed and implemented to improve root causes of negative social health factors, thereby improving the social health score for a location.

For example, the system identifies a lacking of access to healthy food options or education from the processed data. The system generates a solution to improve the social health associated with those factors and accesses external resources to provide local organizations a connection to the location. The generated solution may include interventions and improvements associated with the location. For example, the solution may include identifying local organizations that can establish community gardens or after-school programs focused on academic achievement to improve the social health of the location based on the processed data. In various embodiments, the platform may weigh the importance of each data source and evaluation question based on its correlation to specific SDOH factors. This approach allows the platform to tailor its assessments and interventions to address the most significant determinants of health in a given community.

The social health score computation enables the system to assess and quantify the social health of a location by correlating resident evaluation data, community data, and location data with social health factors. In various embodiments, the system determines the social health engagement for a location from the generated social health scores. For example, evaluations provided by the platform might inquire to collect data indicating economic stability (e.g., employment status, income level), education (e.g., highest level of education attained), social and community context (e.g., access to transportation, neighborhood safety), or health behaviors (e.g., exercise frequency, smoking habits).

The platform can evaluate location conditions by collecting data on the physical and operation state of the building and its amenities. This might include factors such as maintenance schedules, repair requests, and energy efficiency ratings. By assessing the physical state and the operation of the location, the system can identify potential correlations between the location social health score and the community social health score. For example, a lack of clean, affordable housing may negatively impact economic stability and overall health outcomes in the location. Accordingly, the system generates a location social health score that provides data driven insights into locations where improvements can be made to increase the location social health score for the location. For example, the location social health score can be used to determine interventions and improvements The platform regularly monitors the location's social health score (or social impact score) and adjusts the identified solutions. Accordingly, the system ensures that location managers can identify and improve the social health for their locations to improve the overall community social health score. The system may use the data insights on physical conditions, operations and resident SDOH, to identify interventions and ways to improve. In some embodiments, once the interventions are determined or completed, the system puts the housing provider portfolio and/or resident into an Accelerator.

### Additional Considerations

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied, or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps,” or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, the term “database” can refer to either a body of data, a relational database management system (RDBMS), or to both. As used herein, a database can include any collection of data including hierarchical databases, relational databases, flat file databases, object-relational databases, object-oriented databases, and any other structured collection of records or data that is stored in a computer system. The above examples are example only, and thus are not intended to limit in any way the definition and/or meaning of the term database. Examples of RDBMS' include, but are not limited to including, Oracle® Database, MySQL, IBM® DB2, Microsoft® SQL Server, Sybase®, and PostgreSQL. However, any database can be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In another example, a computer program is provided, and the program is embodied on a computer-readable medium. In an example, the system is executed on a single computer system, without requiring a connection to a server computer. In a further example, the system is being run in a Windows® environment (Windows is a registered trademark of Microsoft Corporation, Redmond, Washington). In yet another example, the system is run on a mainframe environment and a UNIX® server environment (UNIX is a registered trademark of X/Open Company Limited located in Reading, Berkshire, United Kingdom). In a further example, the system is run on an iOS® environment (iOS is a registered trademark of Cisco Systems, Inc. located in San Jose, CA). In yet a further example, the system is run on a Mac OS® environment (Mac OS is a registered trademark of Apple Inc. located in Cupertino, CA). In still yet a further example, the system is run on Android® OS (Android is a registered trademark of Google, Inc. of Mountain View, CA). In another example, the system is run on Linux® OS (Linux is a registered trademark of Linus Torvalds of Boston, MA). The application is flexible and designed to run in various different environments without compromising any major functionality.

In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, an element or step recited in the singular and proceeded with the word “a” or “an” should be understood as not excluding plural elements or steps, unless such exclusion is explicitly recited. Furthermore, references to “example” or “one example” of the present disclosure are not intended to be interpreted as excluding the existence of additional examples that also incorporate the recited features. Further, to the extent that terms “includes,” “including,” “has,” “contains,” and variants thereof are used herein, such terms are intended to be inclusive in a manner similar to the term “comprises” as an open transition word without precluding any additional or other elements.

Furthermore, as used herein, the term “real-time” refers to at least one of the time of occurrence of the associated events, the time of measurement and collection of predetermined data, the time to process the data, and the time of a system response to the events and the environment. In the examples described herein, these activities and events occur substantially instantaneously.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer system for evaluating and improving community health scores for a location, the computer system comprising:
one or more resident devices corresponding to the location;
a resident evaluation generated on each of the resident devices, the resident evaluation comprising one or more evaluation questions to collect resident evaluation data from the one or more resident devices, wherein each of the one or more evaluation questions correspond to one or more social health factors, the resident evaluation data reflecting at least one social determinant of health corresponding to each of the resident devices corresponding to the location; and
a remote system server configured to communicate with the one or more resident devices and one or more external data sources corresponding to the location via an external network, the remote system server comprising one or more processors programmed to:
receive a first element of location data from the external data source relating to the location;
receive the resident evaluation data;
generate a community social health score for the location based at least in part on the location data and the resident evaluation data;
determine at least one location improvement or community service to improve the community social health score; and
cause to be displayed, to a location management platform for the location via a graphical user interface, information about the at least one location improvement and community service.

2. The computer system of claim 1, wherein each of the one or more evaluation questions is assigned a weight corresponding to at least one of the social health factors.

3. The computer system of claim 1, wherein the community social health score is classified into one of a plurality of categories including, but not limited to, success, progressive, distressed, and hazard, based on one or more threshold values.

4. The computer system of claim 1, wherein the one or more processors are further programmed to:
determine, using artificial intelligence and/or machine learning techniques, a predicted decrease in the community social health score; and
transmit an alert to the location management platform advising of the predicted decrease in the community social health score.

5. The computer system of claim 1, wherein the location data comprises at least one of: power usage for the location, water usage for the location, current and past temperatures at the location, or maintenance history for the location, wherein at least some of the location data is provided by sensors and/or smart devices located at the location.

6. The computer system of claim 1, wherein the one or more processors are further programmed to execute a machine learning model trained to predict disruptions in the community social health score based on historical resident evaluation data and location data.

7. The computer system of claim 1, wherein the one or more processors are further programmed to communicate with a service provider platform to match residents with external community service providers.

8. The computer system of claim 1, wherein the graphical user interface comprises a dashboard configured to display a map of residents and facilities with corresponding social health categories.

9. The computer system of claim 1, wherein the location management platform comprises a smart router configured to aggregate data from Internet-of-Things devices at the location and transmit the aggregated data to the remote system server.

10. The computer system of claim 1, wherein the one or more processors are further programmed to recommend infrastructure enhancements including at least one of: development of public spaces, community centers, or transportation services in order to improve the community social health score.

11. The computer system of claim 1, wherein the community social health score is generated as a weighted aggregate of resident evaluation data, location data, and external community data.

12. The computer system of claim 1, wherein the one or more processors are further programmed to anonymize the resident evaluation data prior to generating the community social health score.

13. The computer system of claim 1, wherein the one or more processors are further programmed to:
generate a digital location profile comprising attributes of the location and associated residents; and
update the digital location profile over time based on collected data.

14. The computer system of claim 1, wherein the one or more resident devices access the resident evaluation by scanning a quick response (QR) code, wherein each QR code encodes a unique uniform locator (URL) corresponding to a specific property and evaluation combination.

15. The computer system of claim 1, wherein the one or more processors are further programmed to:
replay historical resident evaluation data;
identify potentially anomalous or malicious responses based on predefined filtering criteria; and
selectively exclude identified responses from the generation of the community social health score.

16. The computer system of claim 1, wherein the one or more processors are further programmed to:
track the community social health score over a plurality of time periods; and
generate a temporal trend analysis, the temporal trend analysis illustrating one or more changes in the community social health score over time and measuring an impact of the at least one location improvement and community service.

17. The computer system of claim 1, wherein the location management platform is further configured to implement a secure authentication flow, the location management platform comprising one or more processors programmed to:
generate a temporary password for a user account;
transmit the temporary password to a registered email address;
prompt a mandatory password reset upon first login with the temporary password; and
log all authentication actions associated with the user account.

18. The computer system of claim 8, wherein the dashboard is further configured to:
display one or more customer-specific branding elements including uploaded logo graphics; and
provide a plurality of drag-and-drop interface tools to customize one or more data visualization components.

19. The computer system of claim 1, wherein the resident evaluation further comprises a Likert scale response format, wherein each response option is associated with a visual indicator graphic.

20. A location management computer platform for collecting data from a resident using a resident computing device and providing the data to a location management service provider, the location management computer platform comprising a resident evaluation, an external data source, and at least one processor in communication with at least one memory device, the at least one processor programmed to:
generate a plurality of evaluation questions for the resident evaluation, each evaluation question corresponding to at least one social health factor;
receive resident evaluation data from a select resident device;
receive location data from at least one location device or the external data source;
generate a community social health score based on the resident evaluation data and the location data; and
generate and post an electronic notification to a dashboard accessible by the location management service provider detailing an improvement for the location identified to increase the community social health score of the location.

21. The location management platform of claim 20, wherein the at least one processor is further programmed to assign a weight to each of the plurality of evaluation questions, each weight corresponding to at least one of the social health factors.

22. The location management platform of claim 20, wherein the at least one processor is further programmed to classify the community social health score into one of a plurality of categories including, but not limited to, success, progressive, distressed, and hazard, based on one or more threshold values.

23. The location management platform of claim 20, wherein the at least one processor comprises a machine learning model trained to generate the plurality of evaluation questions and to predict disruptions in the community social health score based on historical resident evaluation data and location data.

24. The location management platform of claim 20, wherein the electronic notification comprises a graphical user interface element configured to display a map of residents and facilities with corresponding social health categories.

25. A computer implemented method for evaluating and improving community health scores for a location, the method implemented using a computer system having one or more processors, the method comprising:
generating, for display on one or more resident computing devices associated with the location, a resident evaluation including one or more evaluation questions, each evaluation question corresponding to at least one social health factor;
receiving, by the one or more processors, resident evaluation data from the one or more resident computing devices, the resident evaluation data reflecting at least one social health factor associated with the location;
receiving, by the one or more processors, location data of at least one external data source; and
generating, by one or more processors, a community social health score for the location based at least in part on the resident evaluation data and the location data.

26. The method of claim 25, wherein generating the resident evaluation and generating the community social health score are performed by a machine learning model trained on historical resident evaluation data and location data.

27. The method of claim 25, wherein generating the resident evaluation comprises:
generating a language selection interface for display on the one or more resident computing devices;
receiving, by the one or more processors, a language selection from the one or more resident computing device; and
generating the one or more evaluation questions in the selected language.
